(19) United States
(12) Patent Application Publication
(10) Pub. No.: US 2004/0024308 A1
(43) Pub. Date: Feb. 5, 2004

(54) Method and apparatus for intracorporeal medical imaging using self-tuned coils

(76) Inventors: Samuel A. Wickline, St. Louis, MO (US); Frank Hockett, St. Charles, MO (US); Anne Marie Morawski, St. Louis, MO (US); Xin Yu, St. Louis, MO (US)

Correspondence Address: THOMPSON COBURN, LLP, ONE US BANK PLAZA, SUITE 3500, ST LOUIS, MO, 63101, US

(21) Appl. No.: 10/454,933

(22) Filed: Jun. 5, 2003

Related U.S. Application Data
(63) Continuation-in-part of application No. 10/210,931, filed on Aug. 2, 2002.

(51) Int. Cl. A61B 5/05

(52) U.S. Cl. 600/422

(57) ABSTRACT

Disclosed herein is an RF probe for use with a medical imaging apparatus, the probe comprising an intracorporeal self-tuned resonator coil for receiving a signal indicative of an image of an interior portion of a body. The resonator coil is preferably self-tuned to a desired frequency according to one of its geometric parameters. According to one embodiment, the resonator coil comprises a base coil having a plurality of turns and an antenna in circuit with the base coil and extending axially outward therefrom, the antenna having a length such that the resonator coil is self-tuned to a desired frequency. The antenna is preferably a monopole. The resonator coil is also preferably self-matching with respect to a transmission medium coupled thereto. Because the preferred resonator coil of the present invention is self-tuning and self-matching, it avoids the use of bulky and relatively expensive tuning and matching circuits.

### CROSS-REFERENCE AND PRIORITY CLAIM TO RELATED APPLICATION

This application is a continuation-in-part of pending U.S. application Ser. No. 10/210,931, filed Aug. 2, 2002, entitled “Method and Apparatus for Intracorporeal Medical Imaging Using A Self-Tuned Coil,” the entire disclosure of which is incorporated herein by reference.

### FIELD OF THE INVENTION

The present invention relates to generating medical images of an internal portion of the body through the use of an imaging probe inserted into the body. More particularly, the present invention relates to improved intravascular RF probes used in conjunction with magnetic resonance imaging (MRI).

### BACKGROUND OF THE INVENTION

MRI imaging has become a widely used and well-known imaging modality for generating images of interior portions of the human body. Because those of ordinary skill in the art are quite familiar with the basic concepts of MRI, those concepts need only be briefly set forth as background for the invention.

Toward that end, as is well-known, MRI machines are used to create images of interior portions of the body. In doing so, an MRI machine applies a magnetic field to at least a portion of the body to be imaged. A typical magnetic field strength is 1.5 T, although other field strengths are used (commonly in the range of 0.5 T-3.0 T). Thereafter, localized gradients are created in the magnetic field, and RF pulses are applied to a target area representing the portion of the body for which an image is desired. A typical frequency for the RF pulse is the Larmour frequency (around 63 MHz for protons in a magnetic field of 1.5 T). Protons in the target area absorb energy from the RF pulse in an amount sufficient to change their spin direction. Once the RF pulse is turned off, the protons release excess stored energy as they return to their natural alignment in the magnetic field. When releasing this stored energy, signals are created that are indicative of an image of the target area. When properly sensed, such signals can be processed by a computer to generate an MR image of the target area.

It is known in the art to receive such signals through the use of an intracorporeal RF probe (also referred to as an RF receiver). When disposed in the body proximate to the target area, such RF probes are capable of sensing the proton emissions and providing the sensed signal to the image generating computer system by way of a transmission medium such as a coaxial cable. Because such probes may be inserted into the body through very small openings, it is important that those receivers have as small of a mechanical envelope as possible.

Also, it is important that the receiver coil resonate (i.e., efficiently store energy) at the Larmour frequency. To resonate a particular frequency f, the inductive components (L) and capacitive components (C) of the receiver coil should satisfy the following equation:

$$f = \frac{1}{2 π \sqrt{L C}}$$

The RF probes in prevalent use for MR imaging can be grouped into two basic categories (1) an elongated coil with a thin cross section, and (2) a loopless antenna (dipole) consisting of a single thin wire. An example of an elongated coil design for an RF receiver is described by Quick et al. in Single-Loop Coil Concepts for Intravascular Magnetic Resonance Imaging, Magnetic Resonance in Medicine, vol. 41, pp. 751-758 (1999), the entire disclosure of which is hereby incorporated by reference. An example of a loopless antenna design is described by Ocali and Atalar in Intravascular Magnetic Resonance Imaging Using a Loopless Catheter Antenna, Magnetic Resonance in Imaging, vol. 37, pp. 112-118 (1997), the entire disclosure of which is hereby incorporated by reference. Other coil examples are Helmholtz coils (which typically consist of two single loop coils in parallel) and flat coils. Further RF probe examples can be found in U.S. Pat. Nos. 4,932,411, 5,715,822, 6,171,240, and 6,453,189, the entire disclosures of all of which are incorporated herein by reference.

FIG. 1 illustrates an exemplary prior art coil receiver assembly. A single loop coil 100 senses the signal emitted by the target area responsive to the RF pulses. Both coil 100 and thin coaxial cable 102 can be disposed inside the body of the patient. The signal passes from coil 100 through thin coaxial cable 102 to thicker coaxial cable 104, which may be RG 58 cable or the like Together the thin and thick coaxial cables 102 and 104 have a length of λ/2 and form part of a tuned resonance circuit. The coil receiver assembly also includes an external tuning/matching circuit 106 as shown, wherein variable tuning capacitor Cₜ forms a resonant circuit with the inductance of the coil 100 and cables 102 and 104, and variable matching capacitor Cₘ matches the input impedance of the resonance circuit with that of the receiver (50 Ω).

FIGS. 2(a) and 2(b) illustrate an exemplary prior art antenna receiver assembly. Dipole antenna 110 is shown in FIG. 2(a). The dipole antenna 110 is formed of two separated conductors 112 and 114. As the current path is not complete, charge oscillates between the two tips of the conductors 112 and 114. When implemented, the antenna 110 is coupled with thin coaxial cable and disposed within a catheter 120. Catheter 120 may be inserted within the body proximate to the target area for imaging thereof. For satisfactory quality of performance, the input impedance of the antenna 110 (ZIN) must be matched with the characteristic impedance of coaxial cable 122 shown in FIG. 2(b). Also, to avoid interference caused by antenna resonation, detuning is needed to electronically damp the receiver's resonance by presenting the coaxial cable to the antenna as a large magnitude impedance. For these purposes, external tuning/matching/decoupling circuit 124 is provided to link the catheter 120 with coaxial cable 122 (which itself terminates at connector 126).

Such prior art receiver assemblies suffer from various shortcomings, namely (1) the single loop coil design exemplified by FIG. 1 works well for near field resolution but not for far field resolution (due to field cancellation occurring at a relatively short distance from the loop)—the near field and far field pertaining to the physical location of the imaging field relative to the receiver, (2) the antenna design exemplified by FIGS. 2(a) and 2(b) works well for far field resolution but not for near field resolution (as determined by the device's geometry which defines a near/far transition zone), (3) each design requires the use of bulky and relatively expensive external matching circuits and tuning circuits, and (4) the coil design of FIG. 1 allows heat to build up as current passes through the coil. While Helmholtz coils and flat coils do not suffer from troubling near/far field transition zones, those coils require the use of external matching and tuning circuits.

Additional coil designs are shown in the article Rivas et al., “In Vivo Real-Time Intravascular MRI”, Journal of Cardiovascular Magnetic Resonance, 4(2), pp. 223-232, 2002 (the entire disclosure of which is hereby incorporated by reference), all of which suffer from the same or similar shortcomings mentioned above.

Therefore, there is a need in the art of medical imaging for RF probes that provide high performance in both near field and the far field imaging. Further, there is a need in the art of medical imaging for RF probes that avoid the incorporation of bulky external electrical components such as matching circuits and tuning circuits, which not only adversely affect the size of the probes' mechanical envelopes but also add to the cost of the receiver.

### SUMMARY OF THE INVENTION

Toward this end, the inventors herein have developed an RF probe for use with a medical imaging apparatus, the RF probe comprising an intracorporeal self-tuned resonator coil. The self-tuning aspect of the present invention is preferably achieved via appropriate selection and configuration of at least one of the resonator coil's geometric parameters. The inventive coil provides excellent performance in both the near field and far field while having a minimal cross-sectional envelope. The inventive coil achieves a desired magnetic field distribution similar to that of a flat coil (thereby eliminating any significant near/far field transition zones) and a small profile similar to that of a loopless dipole design, all without the need for external tuning circuits or external matching circuits.

When the resonator coil is inserted into a patient's body and when RF pulses are applied to the body at a frequency substantially the same as the resonant frequency of the resonator coil, the resonator coil receives a signal responsive to the RF pulses, the signal being representative of an image of an interior portion of the patient's body. In an embodiment wherein the resonator coil comprises a multi-turn base coil in circuit with an antenna, the antenna length is an important factor affecting the resonator coil's resonant frequency. By appropriately setting the antenna's length, the resonator coil of the present invention can be tuned to substantially match the frequency of the RF pulses (such as the Larmour frequency of 63 MHz in a 1.5 T field).

Preferably, the resonator coil is coupled to a transmission medium that passes the signal from the resonator coil to a processor (the processor being configured to process the resonator coil signal to generate the image therefrom). The transmission medium has a characteristic impedance, and to prevent a standing wave from building up in the resonator coil, the resonator coil needs to be substantially self-matching with respect to the transmission medium's characteristic impedance.

Toward this end, the resonator coil preferably utilizes a base coil having a number of turns such that the impedance of the resonator coil conductor is substantially self-matching with the transmission medium's characteristic impedance.

Because the resonator coil of the present invention allows for both self-tuning and self-matching, the bulky and relatively expensive tuning and matching circuits that are found in the prior art are unnecessary. As such, the cross-sectional envelope of the resonator coil of the present invention is greatly improved (minimized), which allows for the use of the present invention to image within hard to reach places, such as the interior of blood vessels.

Further, the resonator coil of the present invention is preferably an open coil. As such, and unlike the closed loop coil designs of the prior art, much less heat will build up in the coil as RF energy is received. Because relatively little heat is built up, the resonator coil of the present invention provides greater patient safety and comfort than prior art coil designs.

Further still, the present invention can be used to not only diagnose medical conditions such as tumors or arteriosclerosis, but it may also be used in connection with interventional treatments to deliver and monitor the delivery of substances such as therapeutic drugs, nanoparticles, genes, contrast agents, or the like into the patient's body. By monitoring the image derived from the resonator coil's received signal, a doctor can assess the substance's delivery into the patient's body and, if necessary, make adjustments to how the substance is delivered in response to the images.

These and other features and advantages of the present invention will be in part apparent and in part pointed out in the following description and referenced figures.

### BRIEF DESCRIPTION OF THE DRAWINGS

FIG. 1 is an illustration of a prior art RF receiver using a single loop coil design;

FIGS. 2(a) and 2(b) are illustrations of a prior art RF receiver using a loopless antenna design;

FIG. 3(a) depicts a first main embodiment of the resonator coil of the present invention;

FIG. 3(b) is an exploded view of the first main embodiment of the resonator coil;

FIGS. 3(c) and 3(d) depict an exploded view of the first main embodiment of the resonator coil coupled to a transmission medium such as a coaxial cable;

FIG. 4(a) depicts the cross-sectional envelope of an unsheathed resonator coil of the first main embodiment;

FIG. 4(b) depicts the cross-sectional envelope of a sheathed resonator coil of the first main embodiment;

FIG. 5(a) is an equivalent circuit model for tuning the first main embodiment of the resonator coil;

FIG. 5(b) depicts the distributed capacitance CD for the first main embodiment of the resonator coil;

FIG. 6 is a graph illustrating resonant frequency as a function of resonator length for the first main embodiment of the resonator coil;

FIG. 7 is a Smith chart depicting measured impedance for an unloaded resonator coil of the first main embodiment;

FIG. 8 is a Smith chart depicting measured impedance for a loaded resonator coil of the first main embodiment;

FIG. 9 is a Smith chart depicting measured impedance for another unloaded resonator coil of the first main embodiment;

FIGS. 10(a) and 10(b) depict approximate impedance matching circuit models for the resonator coil of the first main embodiment;

FIG. 11 is a Smith chart depicting the measured matched impedance for an unloaded resonator coil of the first main embodiment;

FIG. 12 is a graph depicting the return loss for the resonator coil of FIG. 11;

FIG. 13 is a Smith chart depicting the measured matched impedance for a loaded resonator coil of the first main embodiment;

FIG. 14 is a graph depicting the return loss for the resonator coil of FIG. 13;

FIG. 15 a Smith chart depicting the measured matched impedance for the unloaded resonator coil of FIG. 11, wherein a 5 ft coaxial cable is coupled to the resonator coil;

FIG. 16 is a graph depicting the return loss for the resonator coil of FIG. 15;

FIG. 17 a Smith chart depicting the measured matched impedance for the loaded resonator coil of FIG. 13, wherein a 5 ft coaxial cable is coupled to the resonator coil;

FIG. 18 is a graph depicting the return loss for the resonator coil of FIG. 17;

FIGS. 19(a)-(c) illustrate the resonator coil of the second main embodiment of the present invention;

FIG. 20 illustrates an electrical schematic and equivalent circuit model for the resonator coil of the second main embodiment of the present invention;

FIG. 21 illustrates the resonator coil of the resonator coil of the second main embodiment disposed within an insulating sheath;

FIGS. 22(a) and (b) are tables showing the measured impedance as a function of antenna length for an unloaded and loaded resonator coil of the second main embodiment;

FIG. 23 illustrates the resonant frequency response to the number of base coil turns for the resonator coil of the second main embodiment;

FIG. 24 illustrates the measured return loss for the resonator coil of the second main embodiment;

FIG. 25 illustrates the field intensity for the resonator coil of the second main embodiment;

FIGS. 26(a) and (b) illustrate the resonator coil of the second main embodiment with a tip coil;

FIG. 27 illustrates an alternative implementation of the resonator coil of the second main embodiment;

FIG. 28 illustrates an electrical schematic and equivalent circuit model for the alternative resonator coil of the second main embodiment;

FIGS. 29-30 illustrate images produced using the resonator coil of the second main embodiment;

FIGS. 31(a) and 31(b) depict the use of the present invention to image an interior portion of a patient; and

FIGS. 32(a) and 32(b) illustrate examples of the present invention's implementation as a guidewire.

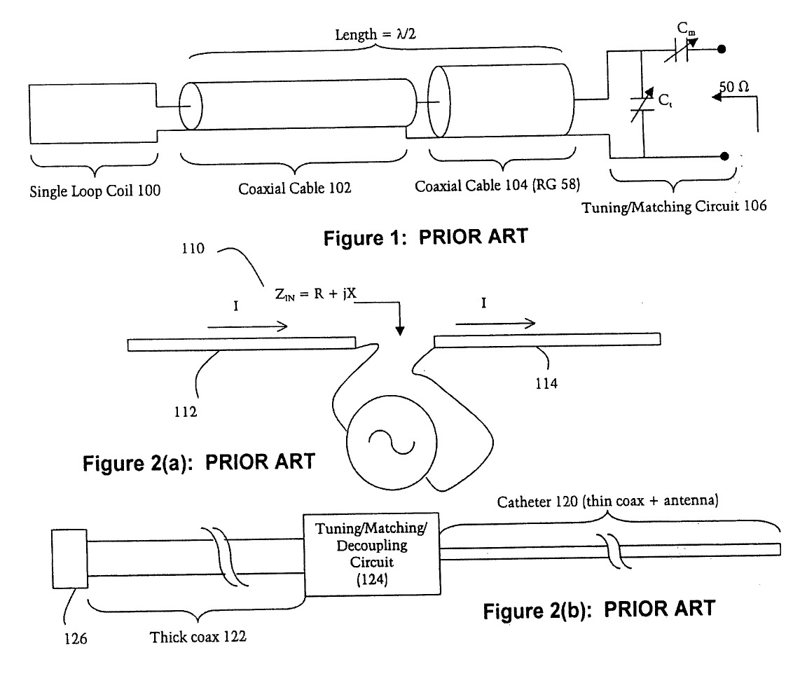

### DETAILED DESCRIPTION OF THE PREFERRED EMBODIMENTS

A. First Embodiment:

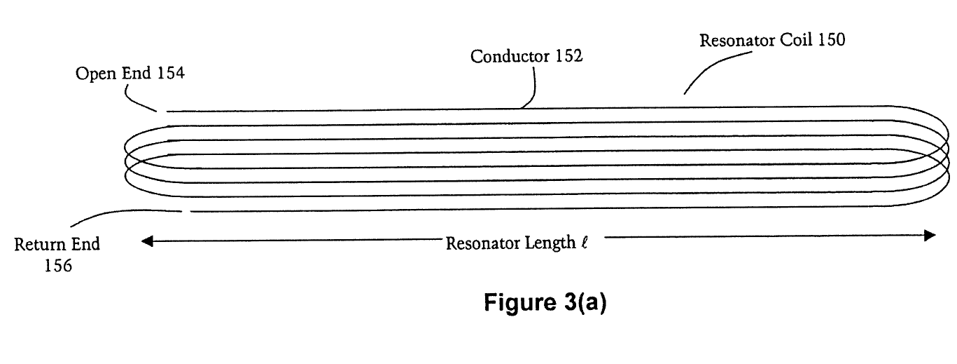

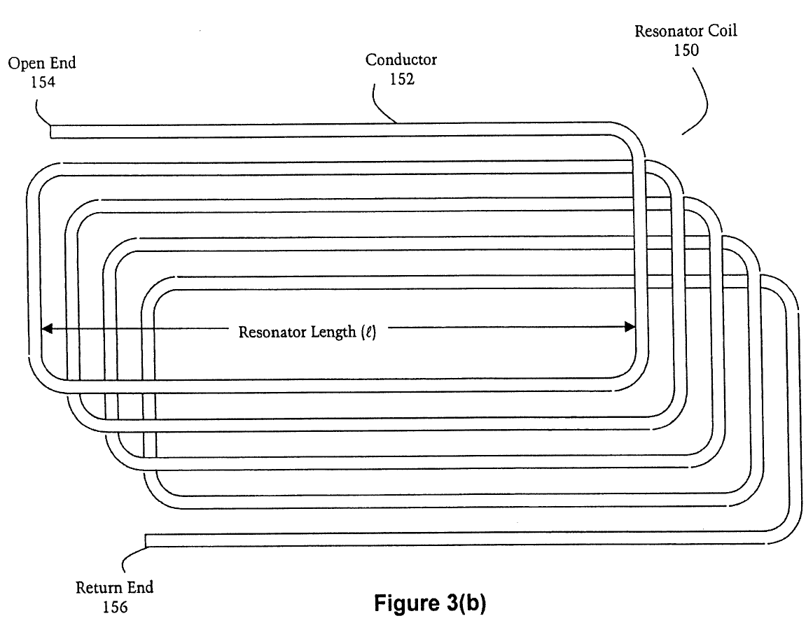

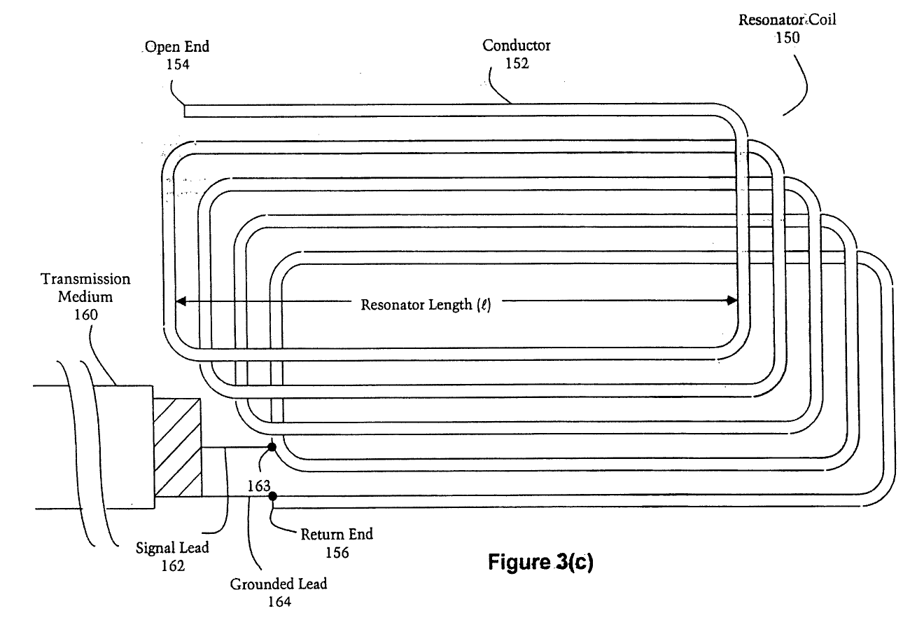

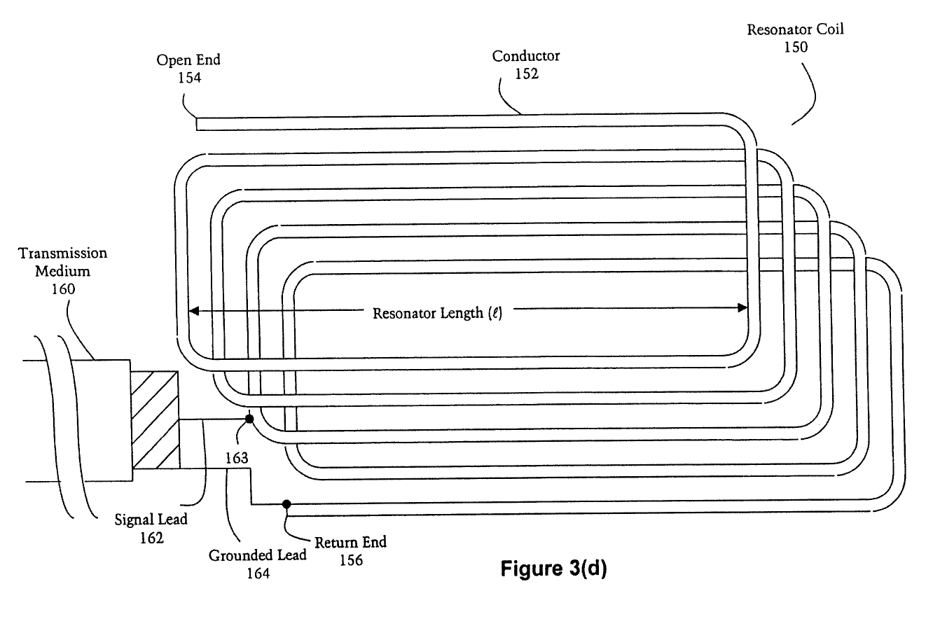

FIG. 3(a) is depicts a first embodiment of the resonator coil 150 of the present invention. Resonator coil 150 is made of a conductor 152 having an open end 154 and a return end 156. Conductor 152 is wound to create a plurality N of turns, thereby forming an open coil. As can be seen, the resonator coil 150 shown in FIG. 3(a) includes 4 turns. However, the actual number of turns that are used for the resonator coil is a design choice, and may be more or fewer than 4, as would be apparent to one of ordinary skill in the art following the teachings of the present invention.

The resonator coil 150 has a length f defined as the length between each turn as shown in FIGS. 3(a)-(d). As will be explained below, the resonator length l is an important factor affecting the resonant frequency of the coil, and the number of turns is an important factor affecting the ability of the coil to be self-matching with the characteristic impedance of a transmission medium connected thereto. FIG. 3(b) is an exploded view of the resonator coil 150, wherein the number of turns is 5.

Conductor 152 is preferably a flexible, small diameter wire such as 30 gauge copper wire or 36 gauge copper wire. However, other gauges of wire reasonably of a similar size may be used, as may non-magnetic wire materials other than copper, as would be apparent to one of ordinary skill in the art. To form the resonator coil 150, the conductor 152 may be hand wound. However, it is preferred that high accuracy industrial winding techniques be used to form a tight winding having a small cross sectional envelope.

The resonator coil 150 is preferably connected to a transmission medium 160 as shown in FIGS. 3(c) and 3(d). Transmission medium 160 passes the signal sensed by the resonator coil 150 to an image processor (not shown). Transmission medium 160 is preferably a flexible small diameter coaxial cable. However, other types of transmission media may be used, such as a shielded twisted pair, as would be apparent to those of ordinary skill in the art.

Transmission medium 160 includes a signal lead 162 and a grounded lead 164. The grounded lead 164 is coupled to the return end 156 of the resonator coil 150. The signal lead 162 is coupled to any intermediate point along any of the turns of the resonator coil. The location 163 of coupling between the signal lead 162 and the resonator coil 150 defines a turns ratio for the resonator coil. The turns ratio is defined as the number of turns in primary winding (the resonator coil 150) to the number of turns in the secondary winding (the winding formed by the coupling of the transmission medium 160 to the resonator coil 150). The turns ratio is an important factor affecting the coil's self-matching capabilities of the resonator coil of the first embodiment, as will be explained below. Referring to FIG. 3(c), it can be seen that the turns ratio is 5:1, while in FIG. 3(d), the turns ratio is 5:2.

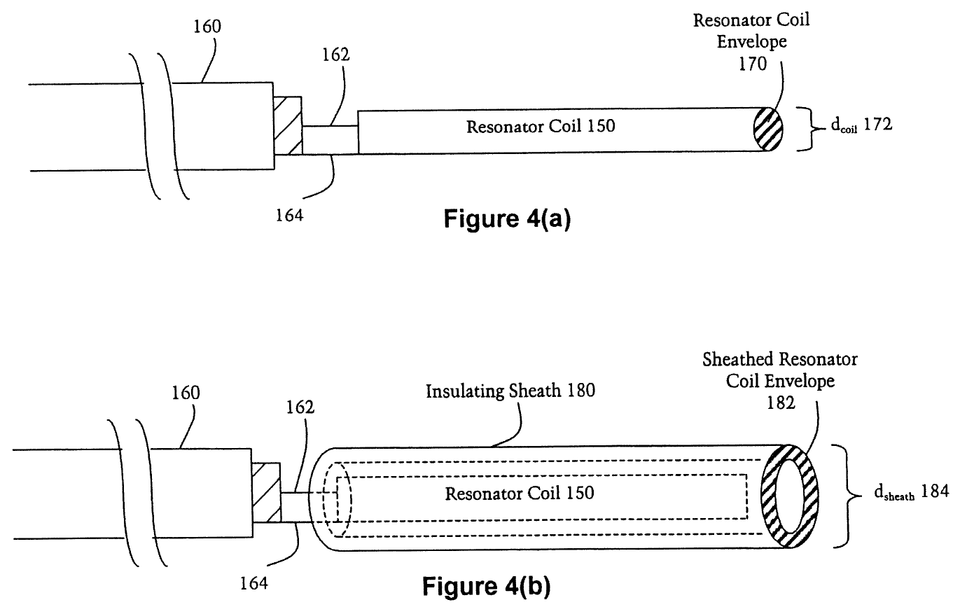

FIG. 4(a) shows the resonator coil (depicted representationally as block 150) coupled to transmission medium 160. The resonator coil 150 has a cross-sectional envelope 170. The diameter 172 (d_{coil}) of the cross-sectional envelope 170 can be sufficiently small to allow insertion of the resonator coil into very minute openings, such as blood vessels or other narrow lumens in the body. Even when the resonator coil 150 is disposed in an insulating sheath 180, as shown in FIG. 4(b), the cross sectional envelope 182 for the sheathed resonator coil is very small. As such, the diameter 184 (dₛₕₑₐₜₕ) is also sufficiently small for insertion of the sheathed resonator coil into minute openings, such as blood vessels or other narrow lumens. With hand wound implementations, the diameter 172 may be as small as 1.2 mm, and diameter 184 may be as small as 2.5 mm, depending upon the gauge of the wire used in the resonator coil 150, the number of turns in the resonator coil 150, and the material used as the sheath 180. Further, it is believed that through the use of manufacturer's microtechnology capabilities, much smaller diameters can be achieved. Given that wound wires used as guidewires with angioplasty balloons can have diameters as small as 0.36 mm, the inventors herein believe that the coil can be as small as 0.25 mm. A preferred range of diameters for the coil of the present invention is 1 mm to 2 mm.

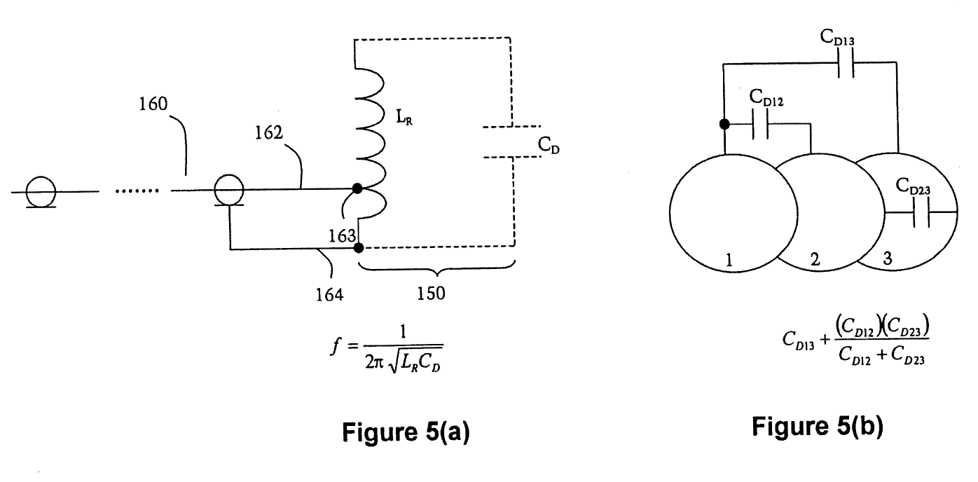

As previously mentioned, one of the advantages of the present invention is its capability to be self-tuned to a desired resonant frequency, thereby eliminating the need for external tuning circuits that are both bulky and relatively costly. FIG. 5(a) illustrates an equivalent circuit model for tuning the resonator coil of the first embodiment. As is well-known, and with reference to the circuit of FIG. 5(a), the resonant frequency of a coil can be expressed by the formula:

$$f = \frac{1}{2 π \sqrt{{L}_{R} {C}_{D}}}$$

wherein L_{R} represents the inductance of the coil and C_{D} represents the distributed (self) capacitance of the coil. See Roddy et al. “Electronic Communications”, 1984, pp 34-35. C_{D} depends upon the resonator's geometry. FIG. 5(b) illustrates how C_{D} is affected when there are 3 coils of wire (coils 1-3). C_{D} can be determined from the individual distributed capacitances shown in FIG. 5(b) as:

$${C}_{D} = {C}_{D13} + \frac{( {C}_{D12} ) ( {C}_{D23} )}{{C}_{D12} + {C}_{D23}}$$

However, while aiding in the understanding of the invention, the formula above is not particularly helpful in tuning the resonator coil because of C_{D}'s high dependence on the resonator's geometry.

The most significant geometrical design factor in self-tuning the resonator coil 150 to a desired resonant frequency, as determined from empirical testing, is resonator length. While other resonator coil properties, such as wire diameter and turns ratio, also have an effect on the coil's resonance, those effects are insignificant. By appropriately selecting the resonator length, and then creating a winding having that length, a practitioner of the present invention can make the self-tuned resonator coil of the first embodiment of the present invention.

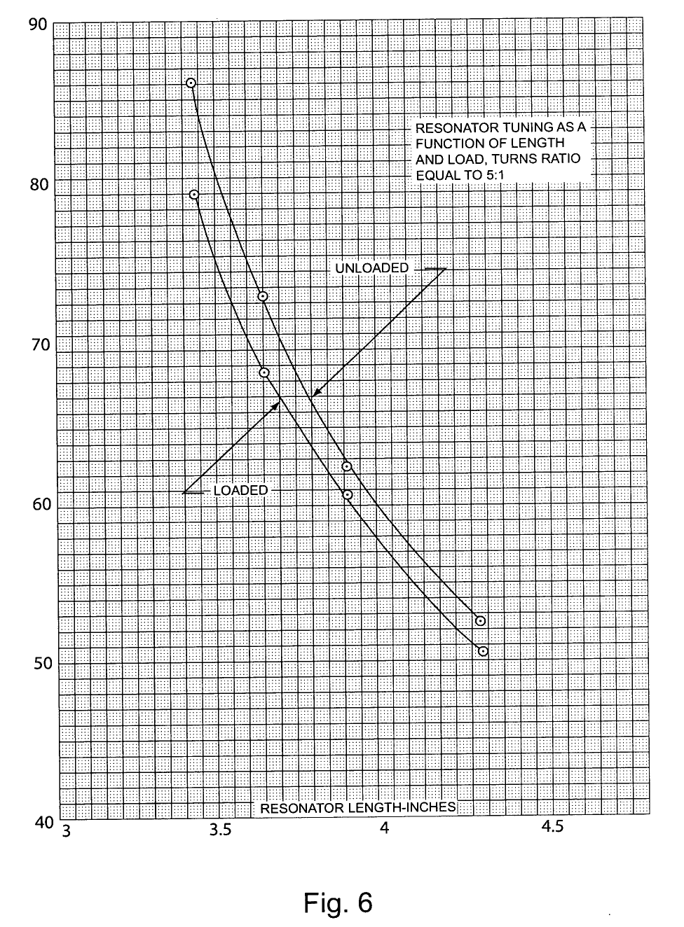

FIG. 6 illustrates resonant frequency as a function of resonator length for a resonator coil formed from 32 gauge copper wire and having a turns ratio of 5:1. FIG. 6 shows plots for both an unloaded resonator coil and a loaded resonator coil. The resonator coil is considered “unloaded” when it is free standing in the air. While there is some dielectric loading from the surrounding air and the enamel paint on the wire, such loading causes negligible energy dissipation (the circuit's Q factor is high). The resonator coil is considered “loaded” once it is encased in an insulating sheath (see FIG. 4(b)), such as heat shrinkable tubing, and immersed in a dielectric. An insulating sheath increases the load on the resonator coil as energy is dissipated into the sheath. Similarly, when subjected to a dielectric (a conductive medium such as saline or the human body), the load on the resonator coil is further increased. The loading referenced in FIG. 6 was achieved by encasing the resonator coil in an insulating sheath and then immersing the sheathed resonator coil in a saline solution.

The data shown in FIG. 6 is reproduced below in Table 1.

TABLE 1
Tuning vs. Resonator Length

| | Resonant | Resonant | % Frequency |
|---|---|---|---|
| | Frequency - | Frequency - | Change |
| Resonator | Loaded | Unloaded | (Unloaded to |
| Length (in) | (MHz) | (MHz) | Loaded) |
| 3.5 | 79.4 | 86.6 | −8.31% |
| 3.65 | 68.2 | 73 | −6.58% |
| 3.9 | 60.2 | 62 | −2.90% |
| 4.3 | 50 | 52 | −3.85% |

A slight curvature exists in this tuning curve. While a linear relationship is expected, the curvature shown in FIG. 6 may be due to variations in the fabrication of different resonator coils used in the experiment, which were hand wound. If a higher quality manufacturing process is used to produce the resonator coil of the present invention, a more linear tuning curve is expected.

Matching the resonator coil of the first embodiment with the characteristic impedance of the transmission medium is primarily a function of resonator length and turns ratio. Because it is preferred that the length of the resonator coil be used to self-tune the resonator coil to a desired frequency, it is also preferred that the turns ratio be used as the variable to self-match the resonator coil with the characteristic impedance of the transmission medium.

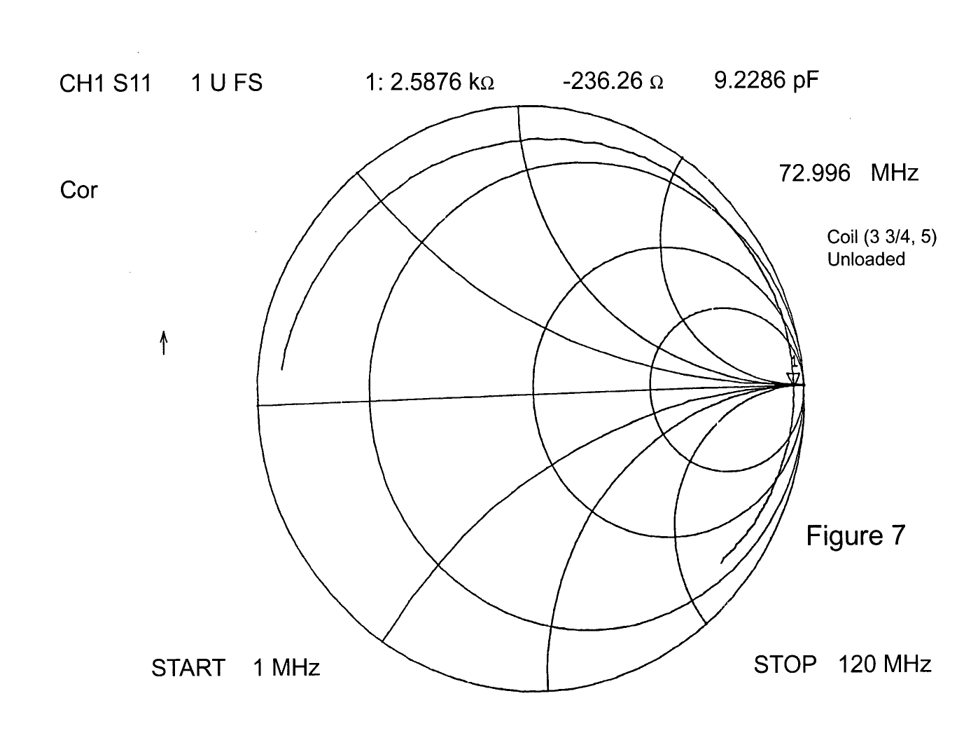

The characteristic load of the resonator coil can be estimated by measuring the reflected impedance of the resonator coil with a network analyzer (for both the loaded and unloaded states). FIG. 7 is a Smith chart illustrating the reflected impedance for an unloaded resonator coil formed from 32 gauge copper wire, having a length of 3¾inches, a 5:1 turns ratio, and a resonant frequency of around 73 MHz. From this figure, it can be seen that the real portion of the coil's load is around 2600 Ω at a low capacitance value of around 9 pF, which is indicative of parallel or high impedance resonance.

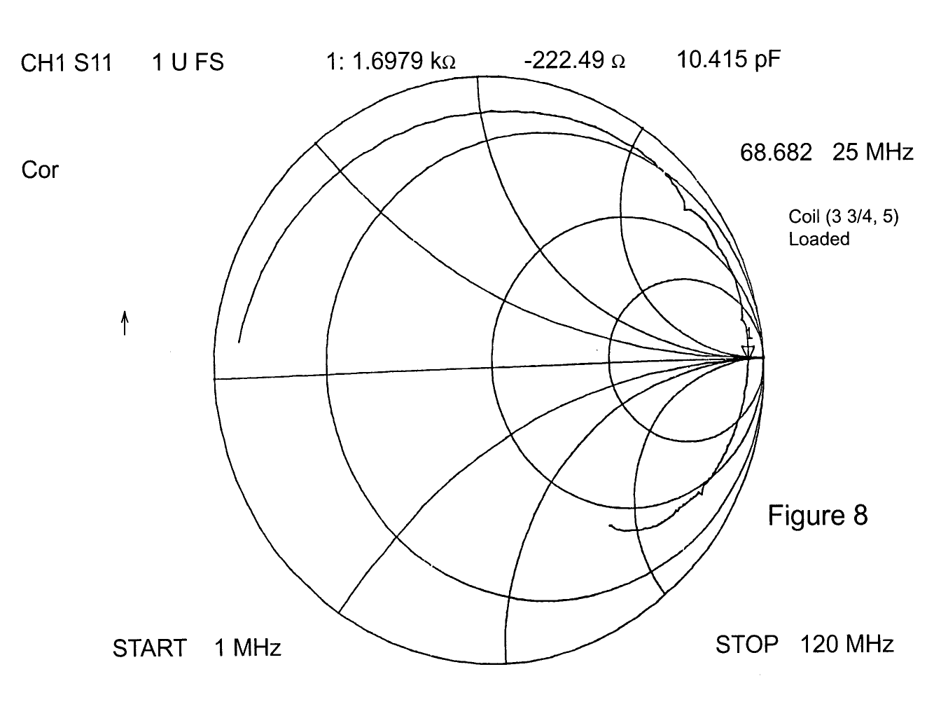

FIG. 8 is a Smith chart illustrating the reflected impedance for a loaded resonator coil of the first embodiment (sheathed and immersed in saline) formed from 32 gauge copper wire, having a length of 3¾ inches, a 5:1 turns ratio, and a resonant frequency of around 68.7 MHz. From this figure, it can be seen that the real portion of the coil's load has decreased to around 1700 Ω.

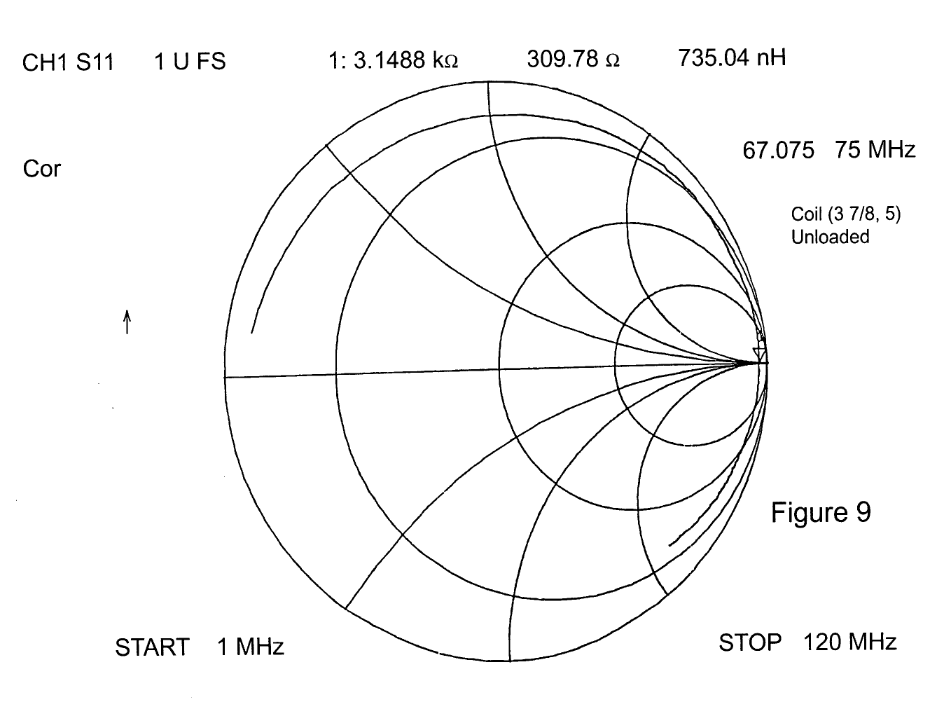

FIG. 9 is a Smith chart illustrating the reflected impedance for an unloaded resonator coil of the first embodiment formed from 36 gauge copper wire, having a length of 3⅞ inches, a 5:1 turns ratio, and a resonant frequency of around 67.1 MHz. From this figure, it can be seen that the real portion of the coil's load is around 3150 Ω.

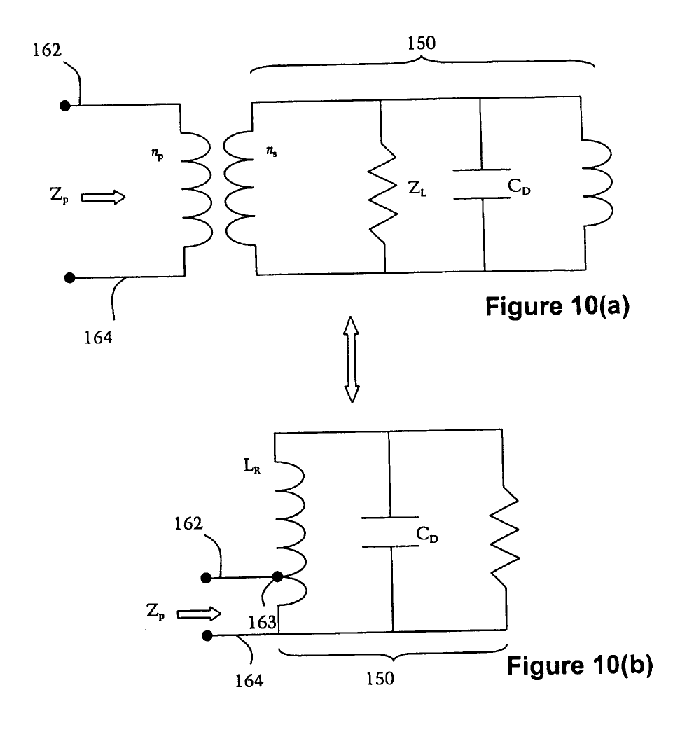

For a resonator coil of the first embodiment having a given length, matching can be achieved through the use of a proper turns ratio. Referring to FIGS. 10(a) and 10(b) which depict an approximate impedance matching circuit model for the resonator coil of the first embodiment, the impedance is reflected through the coil (transformer) as the square of the turns ratio. Thus, for a 5:1 turns ratio, the impedance matching ratio is (5:1)² or 25:1. In the case of the loaded resonator coil used in connection with FIG. 8, the value for Zₚ is (1700 Ω)/(5²) which equals approximately 67 Ω; 67 Ω being a reasonably good match to the 50 Ω characteristic impedance of the transmission medium. To ensure that no significant transmission loss occurs and that no unwanted radiation is present due to mismatching, the voltage standing wave ratio (VSWR) between the resonator coil and the transmission medium should be no greater than 2:1. However, as would be understood by those of ordinary skill in the art, the design parameters for the resonator coil of the first embodiment (resonator length and turns ratio) can be optimized through empirical testing to arrive at a desirably high degree of impedance matching.

While turns ratio has a significant impact on resonator coil matching for the first embodiment, the turns ratio does not have a significant effect on resonator coil tuning. This fact can be explained because the high impedance matching of the present invention provides a high parallel real part (resistance) of the impedance, which does not degrade the resonator coil's Q. For example, for the unloaded and loaded real impedance values of 2700 Ω and 1700 Ω respectively, the resonator coil's Q changes from 90 to 57. For a significant impact on resonator coil tuning, the resonator coil's Q would have to fall to 10 or less.

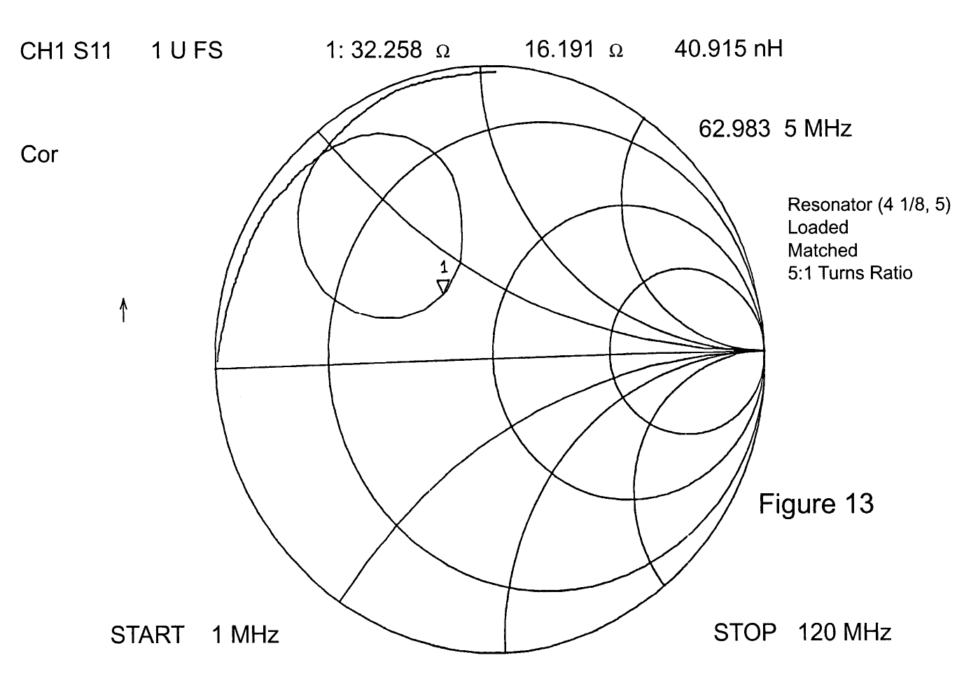

Because turns ratio has an impact on matching, but not tuning (while resonator length has an impact on both tasks), it is relatively easy to both self-tune and self-match the resonator coil of the present invention by first finding a resonator length that tunes the resonator coil of the first embodiment to a desired resonant frequency, and then setting the turns ratio such that the resonator coil substantially matches the characteristic impedance of the transmission medium. To tune a loaded resonator coil to the Larmour frequency (the gyromagnetic ratio of the species to be imaged multiplied by the field strength, which for protons at 1.5 T is around 63 MHz) and match the resonator coil to a 50 Ω transmission medium, a practitioner of the present invention can set the resonator length equal to around 4⅛ inches and the turns ratio equal to 5:1 (see FIG. 13).

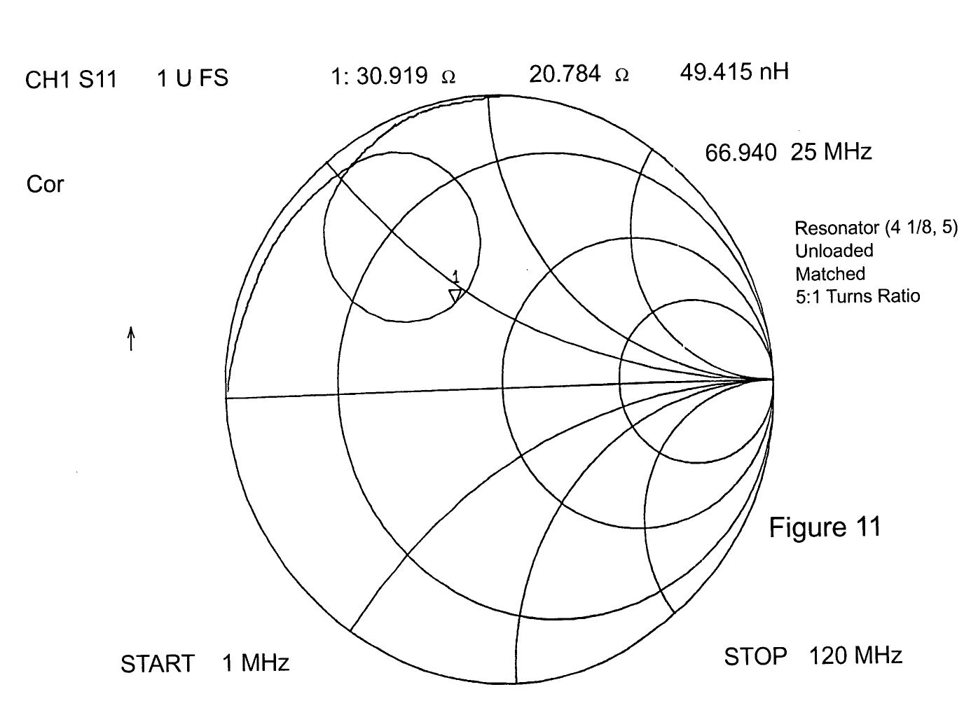

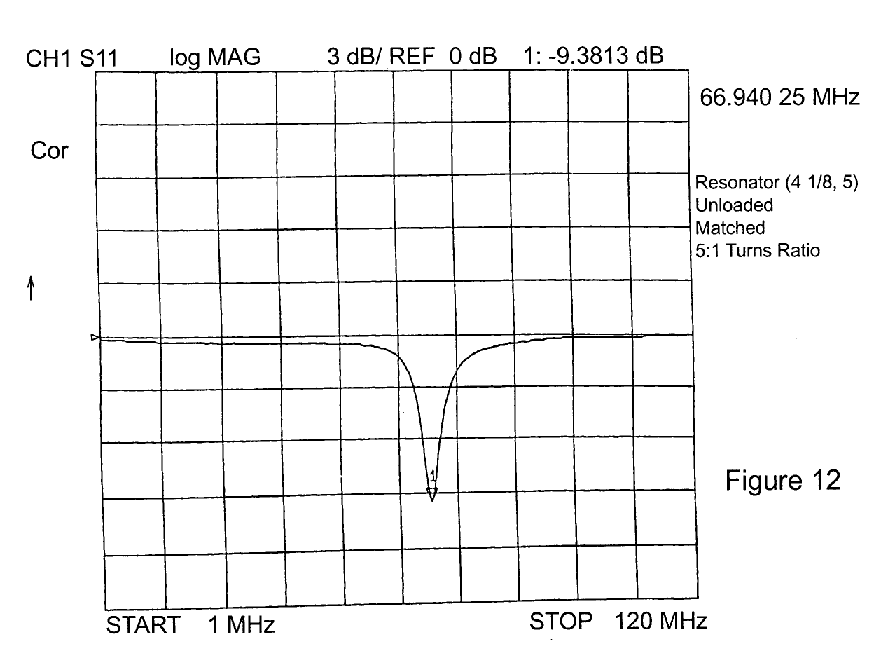

FIG. 11 is a Smith chart depicting a measurement of the matched impedance for an unloaded resonator coil of the first embodiment formed from 32 gauge copper wire, having a length of 4⅛ inches, a 5:1 turns ratio, and a resonant frequency of about 67 MHz. FIG. 12 illustrates the return loss for such a resonator coil. As can be seen, the return loss is about 9.4 dB.

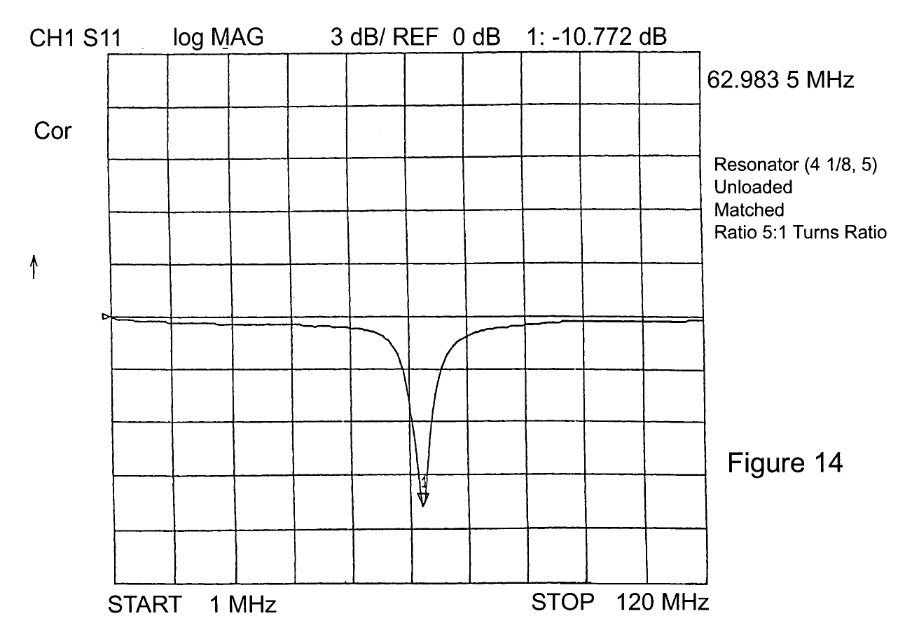

FIG. 13 is a Smith chart depicting a measurement of the matched impedance for a loaded resonator coil of the first embodiment (sheathed and immersed in saline) formed from 32 gauge copper wire, having a length of 4⅛ inches, a 5:1 turns ratio, and a resonant frequency of about 63 MHz. FIG. 14 illustrates the return loss for such a resonator coil. As can be seen, the return loss is about 10.8 dB.

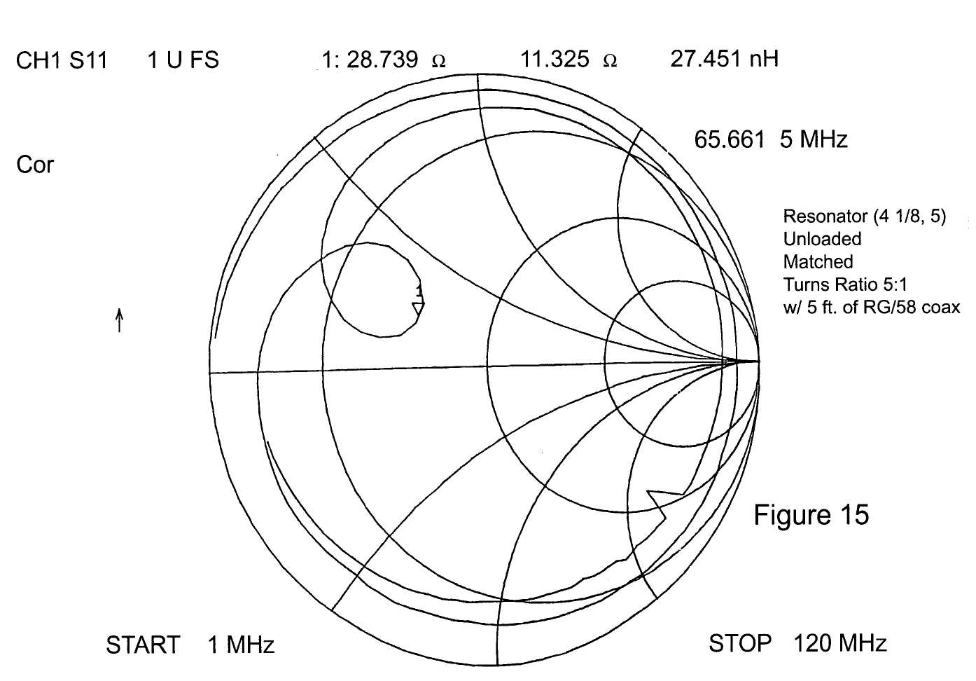

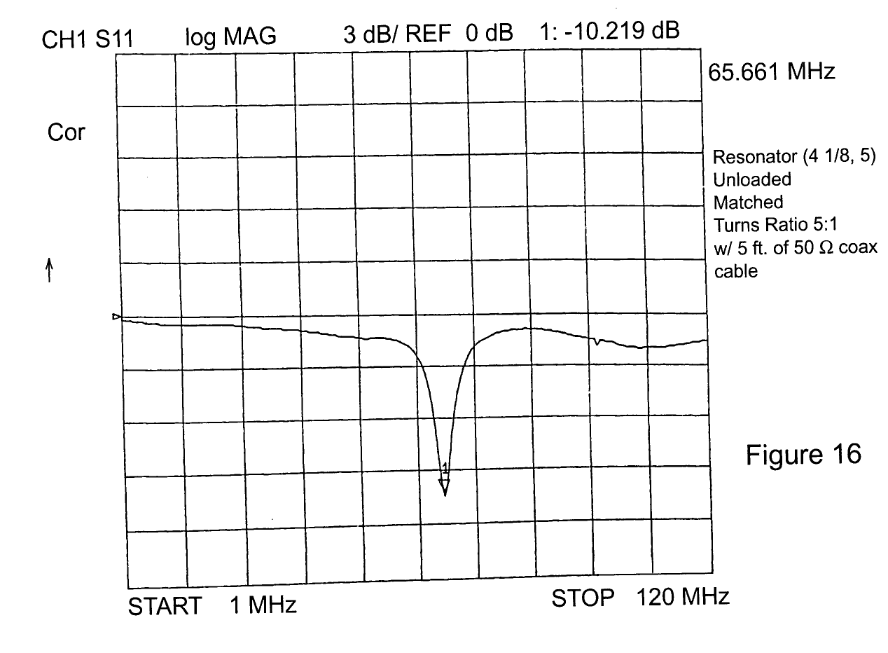

FIGS. 15 and 16 repeat the matched impedance measurement and return loss measurement performed with the unloaded resonator coil of FIGS. 11 and 12, with the exception being that a 5 foot length of RG/58 coaxial cable is coupled to the resonator coil. As can be seen from FIG. 16, the return loss is about 10.2 dB.

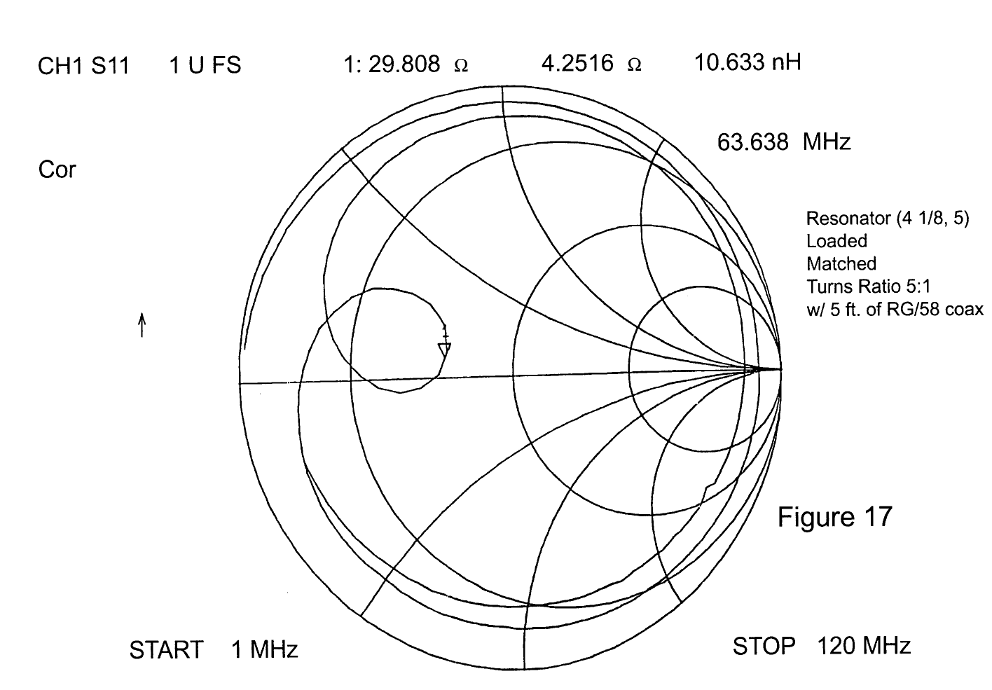

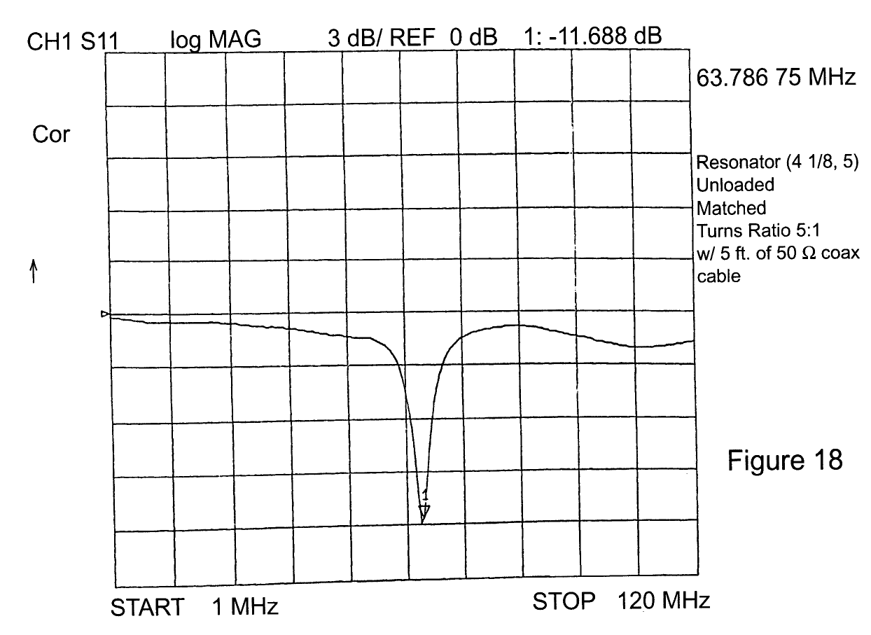

FIGS. 17 and 18 repeat the matched impedance measurement and return loss measurement performed with the loaded resonator coil of FIGS. 13 and 14, with the exception being that a 5 foot length of RG/58 coaxial cable is coupled to the resonator coil. As can be seen from FIG. 18, the return loss is about 11.7 dB.

FIGS. 13-18 show that the resonator coil of the first embodiment is well-behaved when loaded and also suitably matched to the 50 Ω transmission medium, maintaining at least a 10 dB loss for both short and long coaxial cable configurations.

B. Second Embodiment:

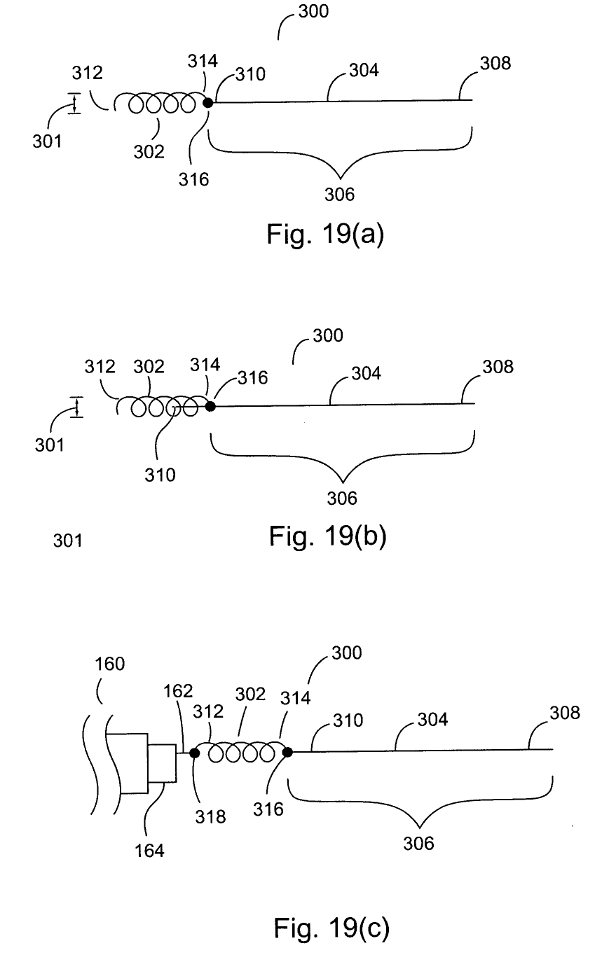

FIG. 19(a) depicts the probe of the second main embodiment of the present invention. As shown, resonator coil 300 of the second embodiment comprises a base coil 302 having a plurality N of turns and an antenna 304 in circuit therewith. The base coil 302 has a proximal end 312 and a distal end 314. The antenna 304 also has a proximal end 310 and a distal end 308. The resonator coil 300 of the second embodiment not only possesses the advantages of the first embodiment over the prior art, but, relative to the first embodiment, the elegantly simple design of the second embodiment allows implementation with ever smaller cross-sectional diameters and can be more easily manufactured. Further, relative to the first embodiment, the second embodiment's ability to be implemented with a smaller cross-sectional envelope allows for easier integration with a catheter, which particularly aids applications where substances are delivered to the patient's body via the catheter. Further still, relative to the first embodiment, the second embodiment provides superior imaging of areas that are a farther orthogonal distance from the resonator coil.

Preferably, the base coil distal end 314 is coupled to the antenna proximal end 310 at coupling point 316. However, it is to be understood that the resonator coil 300 can be implemented such that any point along the base coil distal end portion is coupled to any point along the antenna proximal end portion, wherein the end portion encompasses the actual end point or points nearby. It is preferred that the coupling between the base coil and the antenna be at a point within 0.25 inches of the base coil distal end point and the antenna proximal end point. Further, while early prototypes of the resonator coil 300 are assembled from a separate base coil 302 and antenna 304, it should be understood that the resonator coil 300 can also be formed from a single flexible conductor whose proximal end portion is adapted to form a multi-turn coil and whose distal end serves as the antenna. In fact, it is believed that the use of a single flexible conductor in creating the resonator coil represents the best long-term solution for integrating the resonator coil into a guidewire assembly.

The base coil is preferably formed from a multi-turn solenoidal winding of a flexible conductor. Preferably, the flexible conductor has a small diameter. A preferred range of cross-sectional diameters for the conductor from which the base coil is formed is from approximately 0.1 mm to approximately 0.16 mm. However, a thicker conductor may be used. A preferred conductive winding material is silver-plated (SP) 36 gauge copper wire (or smaller). As will be explained below, the number of coil turns is an important geometric parameter affecting the self-matching capabilities of the resonator coil 300 with respect to a transmission medium that is coupled thereto.

Further, the cross-sectional diameter 301 of the base coil represents the maximum cross-sectional diameter of the resonator coil 300, and is an important factor affecting the suitability of the resonator coil 300 for a variety of medical applications. It is preferred that the diameter 301 be minimized as much as possible to allow for the insertion of the resonator coil 300 into narrow body lumens such as blood vessels. A preferred range of values for diameter 301 extends from approximately 0.3 millimeter (mm) to approximately 1.5 mm. A preferred cross-sectional diameter 301 is one that is less than 0.9 mm. While the experimental resonator coils of the second embodiment produced by the inventors herein possessed a cross-sectional diameter of around 1.5 mm, it should be noted that the base coils for these experimental models were hand-wound and that it is expected that the base coil's cross-sectional diameter can be greatly reduced to the above-described range via any well-known suitable industrial winding technique.

Further still, the base coil preferably has a an axial length that is minimized to the shortest length possible while still retaining the ability to serve as a spatially localizing image artifact. Preferably the artifact comprises two or more voxels in the image, and the base coil axial length may be 10% or less of the monopole length. It is further preferred that the base coil should be wound uniformly with adjacent turns in contact with each other and adjacent layers in contact with each other. However, as would be understood by those of ordinary skill in the art, other axial lengths and less uniform windings can be used in the practice of the present invention.

The antenna 304 is preferably a monopole, and is preferably formed from an elongated small diameter flexible conductor. A preferred conductor material for the monopole 304 is SP 24 gauge copper wire (with a 0.51 mm diameter. A preferred range of acceptable cross-sectional diameters for the monopole 304 extends from approximately 0.3 mm to approximately 1.5 mm. Toward the lower end of this diameter range, a suitable monopole cross-sectional diameter is on the order of 14/1000 of an inch (around 0.36 mm). Also, as will be explained in greater detail below, the monopole length 306 is an important geometric parameter affecting the self-tuning capabilities of the resonator coil 300. The monopole length is defined as the length of the monopole 304 that extends from coupling point 316 to monopole distal end 308. Thus, by appropriately selecting the monopole length 306, the resonator coil 300 can be substantially tuned to a desired frequency such as the Larmour frequency.

FIG. 19(c) illustrates the resonator coil 300 coupled to a transmission medium 160. As noted in connection with the first embodiment, transmission medium 160 preferably includes a signal lead 162 and a grounded lead 164. The preferred transmission medium 160 is a 50 Ω non-magnetic coaxial transmission cable whose diameter is preferably less than 1.5 mm. However, as would be understood by those of ordinary skill in the art, as circumstances justify, larger diameter coaxial cables may be used in the practice of the invention. It is expected that the resonator coil 300 would be used with a length of coaxial cable of approximately 3-5 feet. However, as would be understood by those of ordinary skill in the art, the transmission medium length may be a value outside this range.

The transmission medium 160 exhibits a characteristic impedance, which for the preferred transmission medium of coaxial cable is 50 Ω. The resonator coil 300 is preferably self-matching with respect to this characteristic impedance.

Preferably, the signal lead 162 of the transmission medium 160 is coupled to the proximal end portion of the base coil 302, and more preferably to the proximal end 312 of the base coil 302. The transmission medium serves to carry the signal sensed by the resonator coil 300 to a processor (not shown) associated with a medical imaging apparatus, wherein the processor is configured to generate an image from the received resonator coil signal. A preferred medical imaging apparatus and associated processor for use with the present invention is a 1.5 T MRI imager that permits attachment of RF coils and is capable of digitizing and scan-converting the data received from by the RF coil. However, as would be understood by those of ordinary skill in the art, any MRI imager that permits attachment of RF coils and is capable of digitizing and scan-converting signal data may be used in the practice of the present invention. For example, the present invention may be used with imager field strengths that are higher or lower than 1.5 T, and can be used for nuclei other than protons. As noted below, the present invention is suitable for use with imaging modalities for all MRI visible species, and can also be used for spectroscopy analysis.

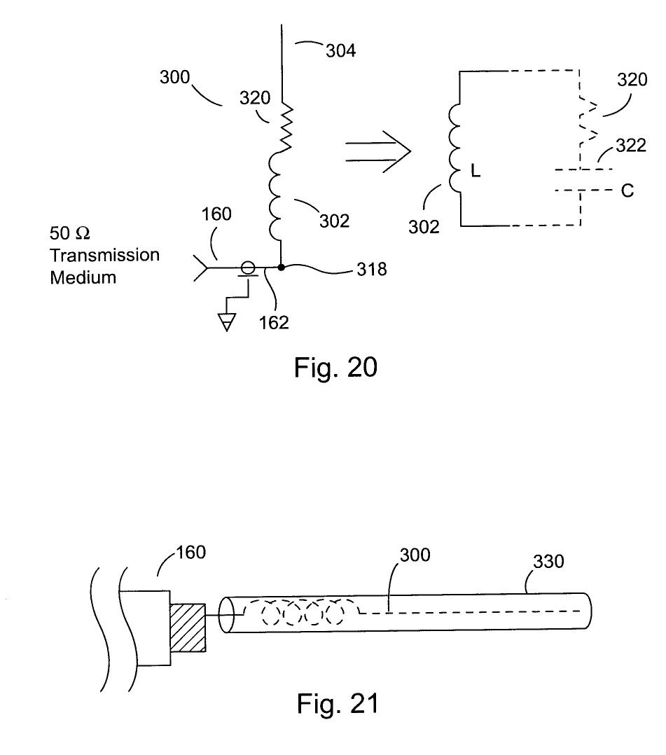

FIG. 20 depicts an electrical schematic and equivalent circuit for the resonator coil of FIG. 19(c), wherein resistance 320 represents the resonator coil loading providing by the imaging sample, such as the patient's body, and wherein capacitance 322 represents the distributed self-capacitance of the resonator coil 300 when the resonator coil is immersed in the imaging body. FIG. 21 illustrates the resonator coil 300 disposed within an insulating sheath, as noted in connection with the first embodiment.

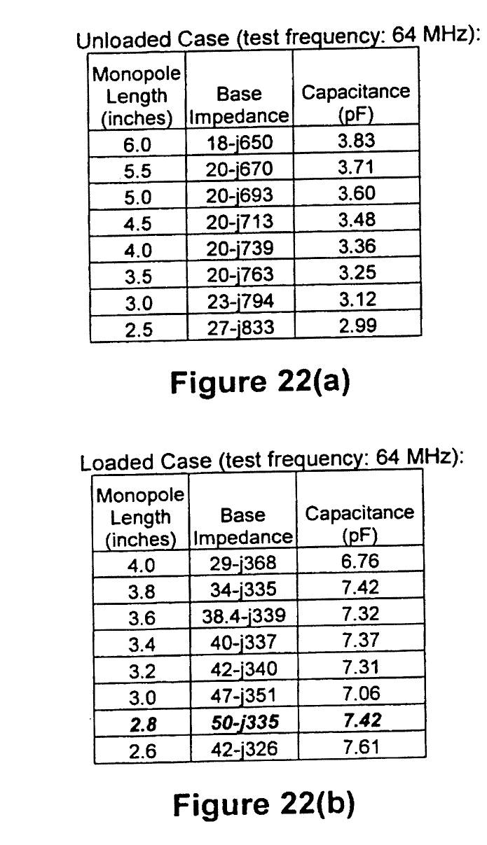

As noted above, the monopole length 306 is an important factor used to tune the resonator coil 300 to a desired frequency. While other geometric parameters of the resonator coil affect resonance (such as the base coil and monopole cross-sectional diameters, base coil material, monopole material, the number of base coil turns), the inventors herein have found monopole length to be the most significant tuning parameter. The table of FIG. 22(a) shows the effect of monopole length 306 on resonance for a monopole 304 formed of 24 gauge SP copper wire having the specified monopole lengths and a cross-sectional diameter of approximately 0.51 mm. As can be seen, for the unloaded case, the various lengths of the monopole simply operate as an electrically short stub antenna with a low value of resistance and a high capacitive reactance that corresponds to approximately 3.5 pF.

However, as shown in FIG. 22(b), when the monopole 304 is loaded by immersion into a saline solution that closely approximates the dielectric properties of the human body, the real part of the measured impedance transitions through 50 Ω for the varying monopole lengths. At a test frequency of the desired tuning frequency of 64 MHz, the monopole 304 becomes resonant for a monopole length of approximately 2.8 inches. While a monopole length of 2.8 inches for tuning the resonator coil 300 to approximately 64 MHz is preferred, it should be understood that the resonator coil can be deemed tuned if the monopole length is one such that a one-port reflection return loss measurement referenced to a nominal 50 Ω real impedance is greater than or equal to 10 dB, or alternatively, that the locus of impedance points lie within a 2:1 VSWR circle on a normalized 50 Ω Smith transmission chart.

Once the appropriate monopole length for tuning the resonator coil 300 to a desired frequency has been chosen, the base coil 302 can be configured to substantially match the resonator coil's impedance with that of the transmission medium by selecting a number of base coil turns sufficient to remove the reactive component of the resonator coil's measured impedance (which for the example of FIG. 22(b) is 335 Ω). Thus, by selecting a sufficient number of turns for the base coil such that the base coil's inductance cancels out the reactive portion of the coil's measured impedance (the relation between coil inductance and the number of coil turns being a well-known in the art), the resonator coil 300 can be made self-matching with respect to the transmission medium. For the example wherein a 2.8 inch monopole is used, the number of base coil turns needed is a number sufficient to create an inductance that resonates with the −j335 Ω. This number comes out to be 66 turns. However, as noted above, the number of turns needed for a substantial match can vary such that the resultant VSWR stays at 2:1 or better (a return loss of around −10 dB). For a 2.8 inch monopole, a satisfactory range of base coil turns is from 65 turns to 70 turns.

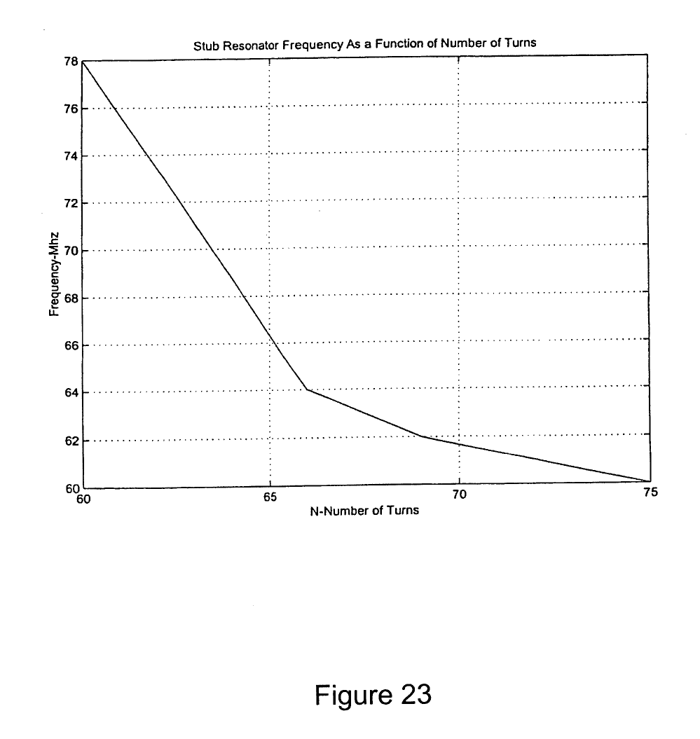

The table below, which is graphically illustrated by FIG. 23, describes resonant frequency response to the number of base coil turns:

TABLE 2
Resonant Frequency Response
to Base Coil Turns

| | Monopole | Base | |
|---|---|---|---|
| | Length | Coil | Frequency |
| Coil Type | (inches) | Turns | (MHz) |
| Coil 1 | 2.8 | 75 | 60 |
| Coil 2 | 2.8 | 60 | 78 |
| Coil 3 | 2.8 | 69 | 61.8 |
| Coil 4 | 2.8 | 69 | 60.5 |
| Coil 5 | 2.8 | 66 | 63.55 |
| Coil 6 | 2.8 | 62 | 89 |
| Coil 7 | 2.8 | 63 | 70.8 |
| Coil 8 | 2.8 | 69 | 64.5 |

Each coil of Table 2 possesses a monopole length of 2.8 inches. Coil 1, which possesses a 75 turn base coil, exhibits a measured resonant frequency of approximately 60 MHz. Coil 2, which possesses a 60 turn base coil, exhibits a resonant frequency of approximately 78 MHz. Coil 3, which possesses a single-layered base coil of 69 turns, exhibits a resonant frequency of approximately 61.8 MHz. Coil 4, which possesses a multi-layered base coil of 69 turns, exhibits a resonant frequency of approximately 60.5 MHz. Coil 5, which possesses a 66 turn base coil, exhibits a resonant frequency of approximately 63.55 MHz. Coil 6, which possesses a 62 turn base coil and a 22 turn tip coil disposed on the distal end portion of the monopole, exhibits a resonant frequency of approximately 89 MHz. Coil 7, which possesses a 63 turn base coil and a 10 turn tip coil disposed on the distal end portion of the monopole, exhibits a resonant frequency of approximately 70.8 MHz. Coil 8, which possesses a 66 turn base coil and a 10 turn tip coil disposed on the distal end portion of the monopole, exhibits a resonant frequency of approximately 64.5 MHz. As can be seen from this data and from FIG. 23, over a large change in base coil turns, the tuning curve is relatively linear. This coincides with the following derivation.

First, assuming that the resonator coil 300's loaded “Q” value is large compared to 1, the resonant frequency can be approximated as:

$${F}_{resonant} = \frac{1}{2 π \sqrt{L C}}$$

wherein L is the inductance of the loaded resonator coil in henries, and wherein C is capacitance of the loaded resonator coil in farads. Given that the inductance L of a coil is proportional to the number N of turns squared:

L=kN²

wherein k is the proportionality constant, then, for two resonator coils with a number of base coil turns N₁ and N₂ respectively, the inductance attributable thereto reduces to:

$$\frac{{L}_{1}}{{L}_{2}} = {( \frac{{N}_{1}}{{N}_{2}} )}^{2}$$

Assuming that the capacitance is the same for the two resonator coils, then the resonant frequency (F₁) for the resonator coil with N₁ turns relative to the resonant frequency (F₂) for the resonator coil with N₂ turns can be defined as:

$$\frac{{F}_{2}}{{F}_{1}} = \sqrt{\frac{{L}_{1}}{{L}_{2}}}$$

which in terms of coil turns, can be expressed as:

$${N}_{2} = {N}_{1} ( \frac{{F}_{1}}{{F}_{2}} ) ; or {F}_{2} = ( \frac{{N}_{1}}{{N}_{2}} )$$

For higher numbers of turns, the curve's linearity is lost, which may be due in part to the inverse relationship shown above, the loss of winding uniformity for larger numbers of turns, and loading variations that may be due to increased coil turns.

Also, it worth noting that the addition of a tip coil to the resonator coil does not greatly influence the resonator coil's tuning, as the resonant frequency does not substantially change for two resonator coils with a 2.8 inch monopole length and 69 base coil turns, wherein one of the resonator coils includes a tip coil and one of the resonator coils does not (the resonant frequency for the former is 64.5 MHz and 61.8 MHz for the latter).

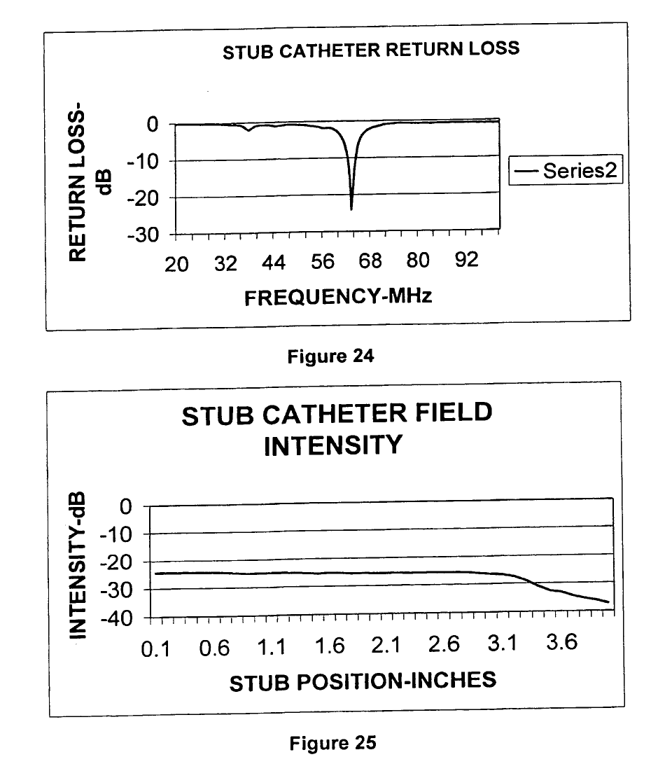

FIG. 24 illustrates the return loss versus frequency for a resonator coil having a monopole length of 2.8 inches and 66 base coil turns. Ideally, the return loss would approach a at the resonant frequency. It is preferred that the return loss be −10 dB or greater to avoid significant signal loss due to mismatching. With the exemplary resonator coil 300 of the present invention, the return loss at 64 MHz (the resonator coil's resonant frequency) is −24 dB, indicating excellent performance.

FIG. 25 charts the field intensity measured for a resonator coil having a monopole length of 2.8 inches and 66 base coil turns at varied positions along the length of the resonator coil, starting from the proximal end of the base coil. As can be seen in FIG. 25, for measurements made at various points lengthwise along the resonator coil, the field intensity is uniform, with some slight fall off as measurements are made beyond the distal end of the monopole. The flat response of the measured field intensity correlates well with the longitudinal sweeps made by MRI machines.

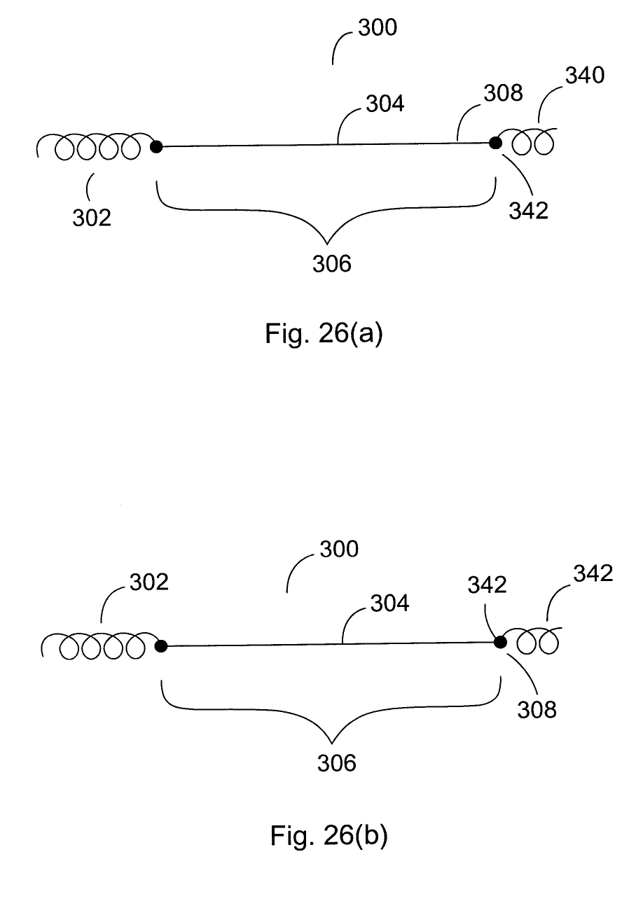

FIG. 26(a) illustrates an implementation of the resonator coil 300 with a tip coil 240 coupled at point 342 to the distal end portion of the monopole 304. As would be understood by those of ordinary skill in the art, the tip coil 340 can be coupled to the distal end 308 of the monopole 304 as shown in FIG. 26(a) or to a point 342 near the monopole's distal end 308 (as shown in FIG. 26(b)). The tip coil shows up in the resultant image as an easily-identifiable artifact, and is thus useful as a navigation aid in locating the distal end 308 of the monopole 302. The typical artifact is also useful as a point for localization. As noted above, the tip coil 340 does not substantially affect the tuning of the resonator coil 300.

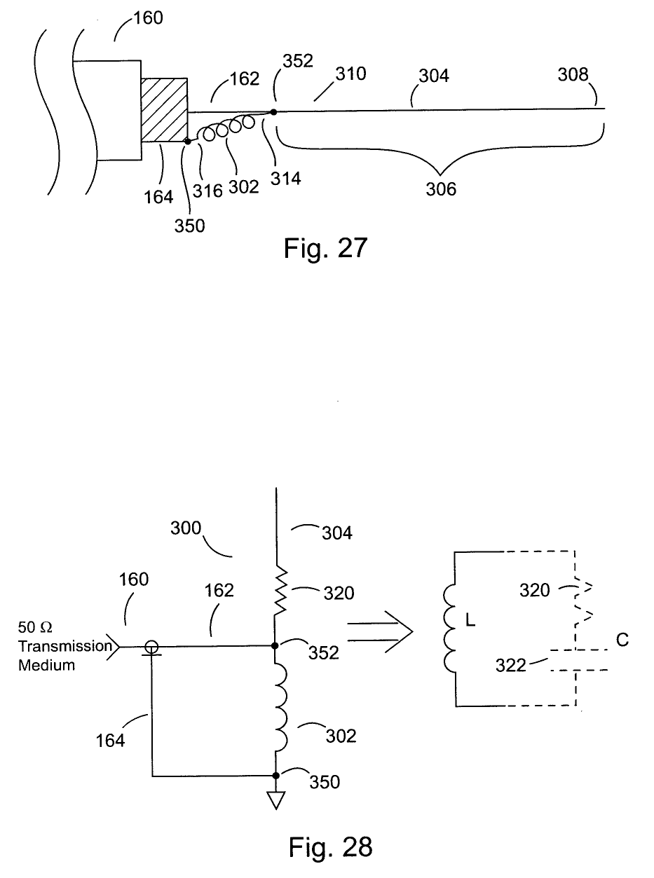

FIG. 27 depicts an alternate coupling of the resonator coil 300 to a transmission medium 160. As shown in FIG. 27, the signal lead 162 of the transmission medium 160 can be coupled to the resonator coil 300 at a point at or near the coupling between the distal end portion of the base coil 302 and the proximal end portion of the monopole 304. Further, the proximal end portion of the base coil 302 is coupled to the grounded lead 164 of the transmission medium 160. The electrical schematic and equivalent circuit model for such a configuration is shown in FIG. 28. The implementation of FIG. 28 may be useful where catheter length considerations will not allow the impedance-to-length relationships (see FIG. 22(b)) to pass through the 50 Ω real part of the impedance.

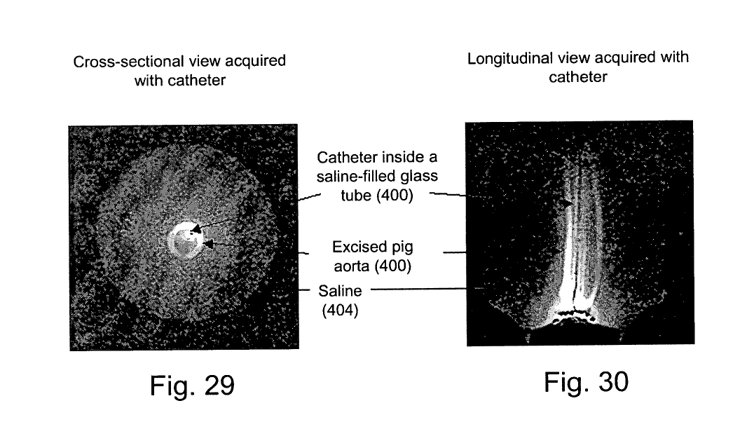

FIGS. 29-30 depict the images produced using a resonator coil 300 having a 2.8 inch monopole and 66 base coil turns in conjunction with a 1.5T clinical magnetic resonance scanner (an NT Intera CV manufactured by Philips Medical Systems of Best, Netherlands) using a T₁-weighted, 2D FFE sequence. The resonator coil 300 was disposed within a catheter and inserted into an excised pig aorta within a saline-filled glass. The catheter 400, pig aorta 402, and saline 404 are all visible in the cross-sectional view of FIG. 29 and the longitudinal view of FIG. 30. The resonator coil's base coil shows up in images as an artifact (not shown), but due to the image field of view in FIG. 30, the base coil artifact is not visible. The base coil artifact (and tip coil artifact, if a tip coil is used, can be useful in passively localizing the catheter while it is inserted within the patient. Further, the base coil artifact is not visible in FIG. 29 as the cross-sectional slice was taken sufficiently far away from the base coil such that the artifact does not show up in the image. FIG. 29 depicts how the resonator coil of the present invention can be used to acquire high resolution images of vessels and vessel walls. FIG. 30, in depicting the longitudinal signal profile of the catheter, provides an indication of the “active” area of the field of view that is, how much of the vessel of interest can be imaged without repositioning the catheter.

C. Applications:

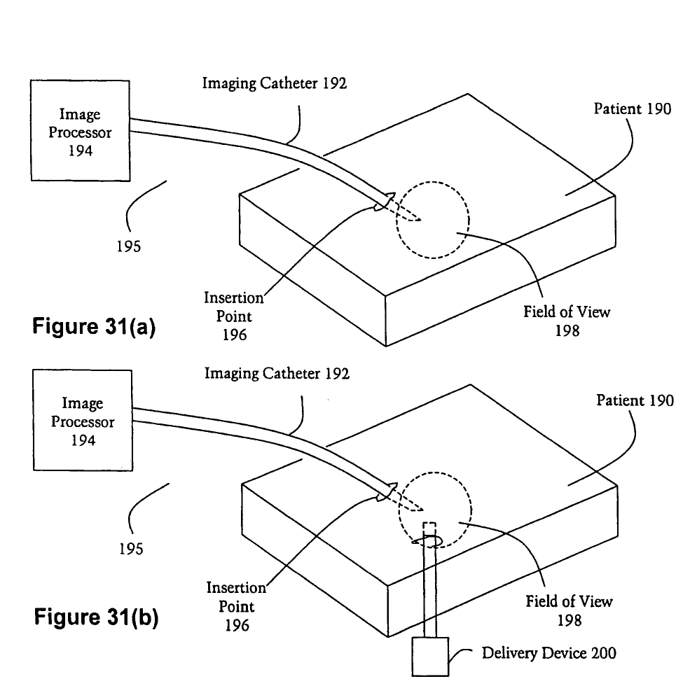

FIGS. 31(a) and 31(b) illustrate how the present invention can be used to image an interior portion of a patient's anatomy. The scope of imaging modalities supported by the coils of the present invention encompasses all MRI visible species, including fluorine sodium, potassium, phosphorus, manganese, carbon, etc., as would be appreciated by those of ordinary skill in the art following the teachings herein. Further, in addition to imaging analysis, the present invention may also be used for spectroscopy analysis.

The medical imaging apparatus 195 shown in FIGS. 31(a) and 31(b) includes the probe of the present invention and transmission medium (which are disposed in the imaging catheter 192) and an image processor 194. The probe is in communication with the image processor 194 via the transmission medium coupled there between. Although the probe is disposed within the imaging catheter 192 in FIGS. 31(a) and (b), this need not be the case as the probe may be used in conjunction with other insertion techniques, as would be readily understood by those of ordinary skill in the art.

Imaging catheter 192 is inserted into the body of patient 190 at insertion point 196. When RF pulses are delivered to the patient's body, the probe will begin receiving a signal that can be translated by the image processor 194 to produce a medical image, such as an MR image, of the interior portion of the patient's body within field of view 198. Due to the probe's small cross-sectional envelope, the probe of the present invention is sufficiently small for insertion into very small openings, such as the coronary artery or a 3 mm artery. As such, the present invention is highly suitable for intravascular imaging to diagnose conditions such as arteriosclerosis (including atherosclerosis), brain imaging to diagnose brain tumors, and MR arthroscopy. The probe of the present invention is also highly suitable for such diagnostic tasks as generating images of the bladder, liver (through insertion into the hepatic vein or artery), pancreas, prostate (through insertion via the urethra), stomach, esophagus, colon, spine, trachea, bronchi, etc.; such images being helpful to determine whether any pathology is present. Further, the probe is also useful for minimally invasive surgery, MR guidance (including the use of passive or active visible elements affixed to the coil containing catheter), interventional MR, and the guidance of surgical instruments.

Further, the probe of the present invention can be used as an imaging guidewire during medical procedures. Most angioplasty guidewires have solid cores with floppy tips, and may (although they usually do not) have a coil wrapped around them, wherein the coils are typically around 0.014 inches in cross-sectional diameter. Most guidewires for larger diagnostic catheters have solid cores that are wrapped with coils up to the very tip, wherein the coils are typically around 0.035 inches in cross-sectional diameter. To use the resonator coil of the present invention as a guidewire, particularly the resonator coil of the second embodiment, it is preferred that a flexible but deformable floppy tip wire portion be affixed to the distal end portion of the monopole. Such a tip wire portion is preferably around 0.5 to 1 inch in length and can be used to cross a tight stenosis in a vessel while still imaging with the resonator coil portion. The imaging guidewire with resonator coil would have to fit within an angioplasty balloon catheter (about a 0.014 inch dimension). Given the small cross-sectional diameter of the present invention, this limitation does not pose a problem. Further, to make an imaging guidewire for advancing a diagnostic catheter, a soft J-tip wire can be affixed to the proximal end portion of the base coil, in which case the resonator coil cross-sectional diameter is preferably around 0.035 inches in diameter.

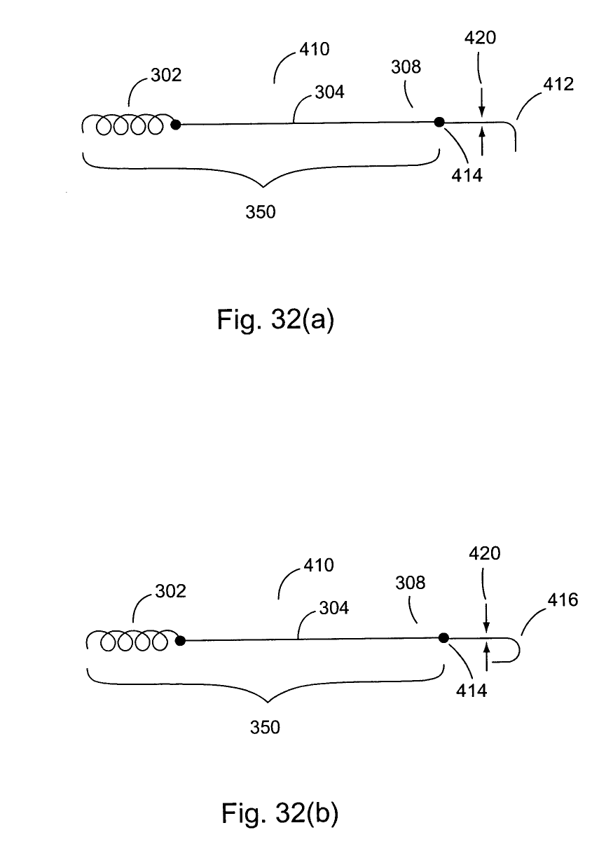

Examples of the present invention's implementation as a guidewire appear in FIGS. 32(a) and (b). In the example of FIG. 32(a), the guidewire 410 comprises the resonator coil 300 with a flexible wire tip 412 coupled at point 414 to the monopole end portion 308. Tip 412 has either a malleable wire that can be shaped by the user, or is preformed into a curve (a hockey stick-like shape in this case) that facilitates navigation through narrowed vessels. Wire tip 412 may have a cross-sectional diameter of approximately 0.014 inches. However, as would be understood by those of ordinary skill in the art, other diameters can be used. In the example of FIG. 32(b), the guidewire 410 comprises the resonator coil 300 with a flexible wire tip 416 coupled thereto at point 414, wherein the wire tip 416 possesses a preformed but flexible candy cane-like shape as might be common with conventional “J-tip” guidewires that are used for advancing diagnostic catheters through larger arteries. Guidewires with a tip 416 as shown in FIG. 32(b) are often used for insertion into the left ventricle. A common cross-sectional diameter 420 for tip 416 is 0.035 inches. However, as would be understood by those of ordinary skill in the art, other diameters and tip configurations may be used.

Further still, as shown in FIG. 31(b), the probe of the present invention can be used as an adjunct to the delivery of substances such as therapeutic drugs, nanoparticles, polymers (including dendrimers), contrast agents, mixtures of materials with contrast agents, genes, paramagnetic materials, superparamagnetic materials, ferromagnetic materials, viruses, and the like into the patient's body. As such substances are delivered to the body to a desired location that is preferably proximate to the location of the catheter's distal end, either through a separate delivery device 200 as shown in FIG. 31(b) (which may be any medical device for injecting a substance into the body—needles, catheters, etc.) or through a channel in the catheter 192, the probe of the present invention can provide real-time feedback as to the accuracy of the substance's delivery. As a substance is delivered to the patient's body within the field of view 198 of the probe, the probe receives a signal representative of that portion of the patient's inner anatomy and passes that received signal to the image processor 194. Once the image processor 194 generates a meaningful image from the probe's signal and that image is displayed, a doctor can make an assessment as to whether his/her delivery of the therapeutic substance is accurate. Depending on the outcome of that decision, the doctor can change the location of substance delivery to thereby improve the patient's treatment.

Yet another application for the probe of the present invention is in connection with image-guided angioplasty, wherein an angioplasty balloon is attached around the coil and inserted into a vessel. Further, drug delivery can be achieved through the balloon. If the balloon is porous, nanoparticles (or other paramagnetic agents) could be injected through the balloon as the balloon is expanded within the vessel. In such cases, the probe could be used simultaneously to visualize the delivery of nanoparticles (or other paramagnetic agents) through the balloon into the vessel or tissue.

Further still, the resonator coil of the present invention can be used for imaging in conjunction with RF ablation procedures, wherein the resonator coil itself is used to deliver high frequency RF pulses to tissue. In such implementations, it is expected that resonator coils having a larger cross-sectional envelope will be used. With this application, the resonator coil will also be coupled to a generator While the resonator coil is not being used to image, the generator can be used to generate high frequency RF pulses that are delivered to a patient's tissue via the resonator coil that is inserted within the patient's body. These RF pulses are useful for cauterization, treatment of heart arrythmia, treatment of brain tumors, and other applications as would be understood by those of ordinary skill in the art.

While the present invention has been described above in relation to its preferred embodiment, various modifications may be made thereto that still fall within the invention's scope, as would be recognized by those of ordinary skill in the art. Such modifications to the invention will be recognizable upon review of the teachings herein. As such, the full scope of the present invention is to be defined solely by the appended claims and their legal equivalents.

What is claimed is:

1. An RF probe for use with a medical imaging apparatus, said RF probe comprising an intracorporeal resonator coil that is self tuned according to at least one of its geometric parameters.

2. The probe of claim 1 wherein the resonator coil comprises:
a base coil having a plurality of turns; and
an antenna in circuit with the base coil and extending axially outward therefrom, the antenna having a length such that the resonator coil is self-tuned to a desired frequency.

3. The probe of claim 2 wherein the antenna is a monopole.

4. The probe of claim 3 wherein the desired frequency is a frequency of substantially the Larmour frequency.

5. The probe of claim 4 wherein the monopole length is approximately 2.8 inches.

6. The probe of claim 5 wherein the number of base coil turns is a number such that the voltage standing wave ratio (VSWR) of the probe, when coupled to a transmission medium, is 2:1 or smaller.

7. The probe of claim 4, wherein the base coil has a proximal end portion and a distal end portion, wherein the monopole has a proximal end portion and a distal end portion, and wherein the proximal end portion of the monopole is coupled to the distal end portion of the base coil, the probe further comprising a transmission medium coupled to the proximal end portion of the base coil, the transmission medium being adapted to pass a signal from the resonator coil to a processor.

8. The probe of claim 7 wherein the transmission medium has a characteristic impedance, and wherein the resonator coil is configured to substantially self-match the transmission medium's characteristic impedance.

9. The probe of claim 8 wherein the resonator coil substantially self-matches the transmission medium's characteristic impedance according to a predetermined number of base coil turns.

10. The probe of claim 4, wherein the base coil has a proximal end portion and a distal end portion, wherein the monopole has a proximal end portion and a distal end portion, and wherein the proximal end portion of the monopole is coupled to the distal end portion of the base coil, the probe further comprising a transmission medium coupled to one selected from the group consisting of (1) the distal end portion of the base coil, (2) the proximal end portion of the monopole, and (3) the coupling point between the proximal end portion of the monopole and the distal end portion of the base coil, the transmission medium for passing a signal from the resonator coil to a processor, and wherein the proximal end portion of the base coil is grounded.

11. The probe of claim 10 wherein the transmission medium has a characteristic impedance, and wherein the resonator coil is configured to substantially self-match the transmission medium's characteristic impedance.

12. The probe of claim 11 wherein the resonator coil substantially self-matches the transmission medium's characteristic impedance according to a predetermined number of base coil turns.

13. The probe of claim 3 wherein the resonator coil has a cross-sectional diameter in a range of approximately 0.3 mm to approximately 1.5 mm.

14. The probe of claim 13 wherein the resonator coil cross-sectional diameter is approximately 0.36 mm.

15. The probe of claim 13 wherein the resonator coil cross-sectional diameter is approximately 0.9 mm.

16. The probe of claim 4 further comprising an insulating sheath within which the resonator coil is disposed.

17. The probe of claim 4 further comprising a tip coil in circuit with the monopole, wherein the tip coil is coupled to the distal end portion of the monopole.

18. The probe of claim 4 wherein the monopole comprises a flexible conductor having a cross-sectional diameter in a range of approximately 0.3 mm to approximately 0.9 mm.

19. The probe of claim 18 wherein the flexible conductor is 24 gauge wire.

20. The probe of claim 18 wherein the monopole has a cross-sectional diameter of approximately 0.3 mm.

21. The probe of claim 18 wherein the base coil is formed from a flexible conductor, the base coil flexible conductor having a cross-sectional diameter in a range of approximately 0.1 mm to approximately 0.16 mm.

22. The probe of claim 21 wherein the base coil has a cross-sectional diameter in a range of approximately 0.7 mm to approximately 1.5 mm.

23. The probe of claim 22 wherein the base coil has a turns-to-length ratio of 44 turns per inch.

24. The probe of claim 3 wherein the resonator coil comprises a flexible conductor, the conductor forming the base coil at its proximal end portion and the monopole at its distal end portion.

25. The probe of claim 1 wherein the resonator coil is an intravascular resonator coil.

26. The probe of claim 1 wherein the resonator coil is also self-matching with respect to a transmission medium coupled thereto according to at least one of the resonator coil's geometric parameters.

27. A magnetic resonance imaging (MRI) probe comprising:
a multi-turn coil; and
a flexible conductor coupled to the coil at a point along the coil's distal end portion, and wherein the conductor is of a length such that the probe is substantially tuned to a desired frequency when inserted into a patient's body.

28. The MRI probe of claim 27 wherein no external tuning components are used to tune the MRI probe to the desired frequency.

29. The MRI probe of claim 28 wherein the desired frequency is a frequency of substantially the Larmour frequency.

30. The MRI probe of claim 29 wherein the conductor length is approximately 2.8 inches.

31. The MRI probe of claim 29 further comprising a transmission medium connected between the MRI probe and a processor, wherein the transmission medium is coupled to the proximal end portion of the coil, wherein the transmission medium has a characteristic impedance, and wherein the number of coil turns is chosen to substantially match the transmission medium's characteristic impedance.

32. The MRI probe of claim 31 wherein the MRI probe substantially matches the transmission medium's characteristic impedance without externally connected components.

33. The MRI probe of claim 32 wherein the number of coil turns is in a range of 65 to 70.

34. The MRI probe of claim 32 wherein the coil has a cross-sectional diameter in a range of approximately 0.7 mm to approximately 1.3 mm.

35. The MRI probe of claim 34 further comprising a multi-turn tip coil coupled to the distal end portion of the conductor.

36. The MRI probe of claim 34 further comprising an insulating sheath within which the coil and conductor are disposed.

37. The MRI probe of claim 29 further comprising a transmission medium for passing a signal received from the MRI probe to a processor, wherein the transmission medium is coupled to one selected from the group consisting of: (1) the distal end portion of the coil, (2) the coupling point between the conductor and the coil, and (3) the proximal end portion of the conductor, wherein the transmission medium has a characteristic impedance, and wherein the number of coil turns is a number such that the MRI probe is substantially matched to the transmission medium's characteristic impedance.

38. A method of generating an image of an interior portion of a patient's body, the method comprising:
inserting an RF probe at least partially inside the patient's body, the RF probe comprising an intracorporeal resonator coil that is self-tuned according to at least one of its geometric parameters; and
using the inserted RF probe in conjunction with a medical imaging apparatus to generate an image of an interior portion of the patient's body.

39. The method of claim 38 wherein the resonator coil comprises:
a base coil having a plurality of turns; and
an antenna in circuit with the base coil and extending axially outward therefrom, the antenna having a length such that the resonator coil is self-tuned to a desired frequency.

40. The method of claim 39 wherein the antenna is a monopole.

41. The method of claim 40 wherein the desired frequency is a frequency of substantially the Larmour frequency.

42. The method of claim 41 wherein the monopole length is approximately 2.8 inches, and wherein the number of base coil turns is in a range of 65 turns to 70 turns.

43. The method of claim 41, wherein the base coil has a proximal end portion and a distal end portion, wherein the monopole has a proximal end portion and a distal end portion, and wherein the proximal end portion of the monopole is coupled to the distal end portion of the base coil, the probe further comprising a transmission medium coupled to the proximal end portion of the base coil, the transmission medium for passing a signal from the resonator coil to a processor associated with the medical imaging apparatus.

44. The method of claim 43 wherein the transmission medium has a characteristic impedance, and wherein the resonator coil is configured to substantially self-match the transmission medium's characteristic impedance.

45. The method of claim 44 wherein the resonator coil substantially self-matches the transmission medium's characteristic impedance according to a predetermined number of base coil turns.

46. The method of claim 41 wherein the using step comprises using the inserted RF probe in conjunction with a magnetic resonance (MR) imaging apparatus to generate an MR image of an interior portion of the patient's body.

47. The method of claim 41, wherein the base coil has a proximal end and a distal end, wherein the monopole has a proximal end and a distal end, and wherein the proximal end of the monopole is coupled to the distal end of the base coil, the probe further comprising a transmission medium coupled to one selected from the group consisting of (1) the distal end of the base coil, (2) the proximal end portion of the monopole, and (3) the coupling point between the proximal end of the monopole and the distal end of the base coil, the transmission medium for passing a signal from the resonator coil to a processor associated with the medical imaging apparatus, and wherein the proximal end of the base coil is grounded.

48. The method of claim 40 wherein the inserting step comprises inserting the RF probe at least partially within a blood vessel of the patient.

49. The method of claim 48 wherein the inserting step further comprises inserting the RF probe at least partially within the patient's hepatic artery.

50. The method of claim 48 wherein the inserting step further comprises inserting the RF probe at least partially within the patient's hepatic vein.

51. The method of claim 40 wherein the inserting step comprises inserting the RF probe at least partially within the patient's urethra.

52. The method of claim 40 wherein the inserting step comprises inserting the RF probe at least partially within the patient's bladder.

53. The method of claim 40 wherein the inserting step comprises inserting the RF probe at least partially within the patient's pancreas.

54. The method of claim 40 wherein the inserting step comprises inserting the RF probe at least partially within the patient's esophagus.

55. The method of claim 40 wherein the inserting step comprises inserting the RF probe at least partially within the patient's stomach.

56. The method of claim 40 wherein the inserting step comprises inserting the RF probe at least partially within the patient's brain.

57. The method of claim 40 wherein the inserting step comprises inserting the RF probe at least partially within the patient's trachea.

58. The method of claim 40 wherein the inserting step comprises inserting the RF probe at least partially within the patient's colon.

59. The method of claim 40 wherein the inserting step comprises inserting the RF probe at least partially within a joint of the patient.

60. The method of claim 40 wherein the resonator coil further comprises a tip coil coupled to the distal end portion of the monopole.

61. The method of claim 40 wherein the inserting step comprises inserting a catheter at least partially inside a patient's body, the catheter having the RF probe disposed therein.

62. The method of claim 40 wherein the resonator coil comprises a flexible conductor having a proximal end portion and a distal end portion, wherein the conductor is adapted to form the base coil at its proximal end portion and the monopole at its distal end portion.

63. The method of claim 38, wherein the using step comprises:
actuating a medical imaging apparatus to cause the inserted RF probe to receive a signal representative of an image of an interior portion of the patient's body; and
generating an image from said received signal.

64. A method of delivering a substance into a patient's body, the method comprising:
inserting a catheter at least partially inside a patient's body, the catheter having disposed therein an RF probe that is substantially self-tuned to a desired frequency;
using the probe in conjunction with a medical imaging apparatus to generate at least one image of an interior part of the patient's body;
positioning the probe near a desired location inside the patient's body to which the substance is to be delivered using the at least one generated image; and
delivering a substance proximate to the desired location.

65. The method of claim 64 wherein the RF probe comprises:
a multi-turn coil having a proximal end portion and a distal end portion; and
a flexible conductor having a proximal end portion and a distal end portion, wherein the coil is coupled to the coil at a point along the coil's distal end portion and the conductor's proximal end portion, and wherein the conductor is of a length such that the probe is substantially tuned to a desired frequency when inserted into a patient's body.

66. The method of claim 65 wherein no external tuning components are used to tune the probe to the desired frequency.

67. The method of claim 66 wherein the desired frequency is a frequency of substantially the Larmour frequency.

68. The method of claim 67 further comprising a transmission medium for passing a signal received from the probe to a processor associated with the medical imaging apparatus, wherein the transmission medium is coupled to the proximal end portion of the coil, wherein the transmission medium has a characteristic impedance, and wherein the number of coil turns is a number such that the probe is substantially matched to the transmission medium's characteristic impedance.

69. The method of claim 68 wherein no external matching components are used to match the probe to the transmission medium's characteristic impedance.

70. The method of claim 67 further comprising a transmission medium for passing a signal received from the probe to a processor associated with the medical imaging apparatus, wherein the transmission medium is coupled to one selected from the group consisting of: (1) the distal end portion of the coil, (2) the coupling point between the conductor and the coil, and (3) the proximal end portion of the conductor, wherein the transmission medium has a characteristic impedance, and wherein the number of coil turns is a number such that the MRI probe is substantially matched to the transmission medium's characteristic impedance.

71. The method of claim 66 wherein the substance is selected from the group consisting of (1) a therapeutic drug, (2) nanoparticles, (3) polymers, (4) genes, (5) a contrast agent, (6) mixtures that include a magnetic resonance (MR) contrast agent, (7) paramagnetic materials, (8) superparamagnetic materials, (9) ferromagnetic materials, and (10) a virus.

72. The method of claim 71 wherein the delivering step comprises delivering the substance proximate to the desired location via the catheter.

73. The method of claim 71 wherein the inserting step comprises inserting the catheter at least partially inside one selected from the group consisting of (1) a blood vessel of the patient, (2) the patient's hepatic artery, (3) the patient's hepatic vein, (4) the patient's urethra, (5) the patient's bladder, (6) the patient's pancreas, (7) the patient's esophagus, (8) the patient's stomach, (9) the patient's brain, (10) the patient's trachea, (11) the patient's colon, and (12) a joint of the patient.

74. The method of claim 66 further comprising adjusting the location of substance delivery at least partially in response to generated image feedback.

75. The method of claim 66 wherein the probe further comprises a multi-turn tip coil coupled to the distal end portion of the conductor.

76. The method of claim 66 wherein the medical imaging apparatus is a magnetic resonance (MR) imaging apparatus, and wherein the using step comprises:
applying a plurality of RF pulses to the patient's magnetized body, the RF pulses having a frequency substantially the same as the frequency to which the probe is tuned;
receiving a signal with the probe that is responsive to the RF pulses, the signal being representative of an image of an interior portion of the patient's body within a field of view of the probe; and
generating an image from the received signal.

77. An RF receiver for use in medical imaging comprising:
a multi turn coil; and
a monopole in circuit with the coil, the monopole having a length such that the probe, when inserted in a patient's body, is substantially tuned to a desired frequency without an external tuning circuit.

78. The receiver of claim 77 wherein the multi-turn coil has a number of turns such that the receiver substantially matches the characteristic impedance of a transmission medium coupled to a proximal end portion of the coil.

79. The receiver of claim 78 wherein the desired frequency is a frequency that is substantially the Larmour frequency.

80. The receiver of claim 79 wherein the monopole length is approximately 2.8 inches, and wherein the number of coil turns is in a range of 65 turns to 75 turns.

81. The receiver of claim 79 further comprising a multi-turn tip coil coupled to a distal end portion of the monopole.

82. The receiver of claim 79 wherein the coil and monopole are integrally formed from a single flexible conductor.

83. The receiver of claim 79 further comprising a catheter within which the coil and monopole are disposed.

84. The receiver of claim 79 wherein the receiver is implemented as an imaging guidewire.

85. The receiver of claim 79 wherein the receiver has a maximum cross-sectional diameter in a range of approximately 0.7 mm to approximately 1.5 mm.

86. The receiver of claim 85 wherein the maximum cross-sectional receiver diameter is approximately 0.014 inches.

87. The receiver of claim 85 wherein the maximum cross-sectional receiver diameters is approximately 0.035 inches.

88. A method of identifying a location in a patient's body for delivery of a substance, the method comprising:
inserting a catheter at least partially inside the patient's body, the catheter having disposed therein an RF probe that is substantially self-tuned to a desired frequency;
using the probe in conjunction with a medical imaging apparatus to generate at least one image of an interior part of the patient's body; and
identifying a location within the generated image to which a substance is to be proximally delivered.

89. The method of claim 88 wherein the probe comprises a multi-turn base coil in circuit with a monopole, wherein the monopole has a length such that the probe is self-tuned to substantially the imaging frequency of the medical imaging apparatus.

90. The method of claim 89 wherein the probe is in communication with the medical imaging apparatus via a transmission medium coupled therebetween, and wherein the probe is substantially self-matching with respect to the characteristic impedance of the transmission medium.

91. The method of claim 90 wherein the monopole length is approximately 2.8 inches, and wherein the number of turns in the multi-turn coil is in a range of 65 turns to 70 turns.

92. The method of claim 91 wherein the cross-sectional diameter of the probe is in a range of approximately 0.7 mm to approximately 1.5 mm.

93. The method of claim 88 further comprising delivering a substance proximate to the identified location.

94. The method of claim 93 wherein the substance is selected from the group consisting of (1) a therapeutic drug, (2) nanoparticles, (3) polymers, (4) genes, (5) a contrast agent, (6) mixtures that include a magnetic resonance (MR) contrast agent, (7) paramagnetic materials, (8) superparamagnetic materials, (9) ferromagnetic materials, and (10) a virus.
